SEQUENCE LISTING (39 sequences)

SEQ ID NO: 1 - protein, 22 aa, Artificial Sequence
  Synthetic sequence
GSGATNFSLLKQAGDVEENPGP

SEQ ID NO: 2 - protein, 21 aa, Artificial Sequence
  Synthetic sequence
GSGEGRGSLLTCGDVEENPGP

SEQ ID NO: 3 - protein, 23 aa, Artificial Sequence
  Synthetic sequence
GSGQCTNYALLKLAGDVESNPGP

SEQ ID NO: 4 - protein, 25 aa, Artificial Sequence
  Synthetic sequence
GSGVKQTLNFDLLKLAGDVESNPGP

SEQ ID NO: 5 - protein, 4 aa, Artificial Sequence
  Synthetic sequence
  Misc_feature (2)..(3): Xaa may be any amino acid residue
  Misc_feature (4)..(4): Xaa is a hydrophobic amino acid residue
PXXX

SEQ ID NO: 6 - protein, 5 aa, Artificial Sequence
  Synthetic sequence
  Misc_feature (2)..(3): Xaa may be any amino acid residue
  Misc_feature (4)..(4): Xaa is a hydrophobic amino acid residue
  Misc_feature (5)..(5): Xaa is a Ser or Thr residue
PXXXX

SEQ ID NO: 7 - protein, 6 aa, Artificial Sequence
  Synthetic sequence
  Misc_feature (2)..(2): Xaa is a Lue or Gln amino acid residue
PXGMTS

SEQ ID NO: 8 - protein, 5 aa, Artificial Sequence
  Synthetic sequence
  Misc_feature (2)..(2): Xaa is a Leu or Gln residue
PXGMT

SEQ ID NO: 9 - protein, 7 aa, Artificial Sequence
  Synthetic sequence
ENLYTQS

SEQ ID NO: 10 - protein, 5 aa, Artificial Sequence
  Synthetic sequence
DDDDK

SEQ ID NO: 11 - protein, 4 aa, Artificial Sequence
  Synthetic sequence
LVPR

SEQ ID NO: 12 - protein, 8 aa, Artificial Sequence
  Synthetic sequence
LEVLFQGP

SEQ ID NO: 13 - protein, 10 aa, Artificial Sequence
  Synthetic sequence
CGLVPAGSGP

SEQ ID NO: 14 - protein, 12 aa, Artificial Sequence
  Synthetic sequence
SLLKSRMVPNFN

SEQ ID NO: 15 - protein, 12 aa, Artificial Sequence
  Synthetic sequence
SLLIARRMPNFN

SEQ ID NO: 16 - protein, 12 aa, Artificial Sequence
  Synthetic sequence
SKLVQASASGVN

SEQ ID NO: 17 - protein, 12 aa, Artificial Sequence
  Synthetic sequence
SSYLKASDAPDN

SEQ ID NO: 18 - protein, 12 aa, Artificial Sequence
  Synthetic sequence
RPKPQQFFGLMN

SEQ ID NO: 19 - protein, 12 aa, Artificial Sequence
  Synthetic sequence
SLRPLALWRSFN

SEQ ID NO: 20 - protein, 12 aa, Artificial Sequence
  Synthetic sequence
SPQGIAGQRNFN

SEQ ID NO: 21 - protein, 14 aa, Artificial Sequence
  Synthetic sequence
DVDERDVRGFASFL

SEQ ID NO: 22 - protein, 12 aa, Artificial Sequence
  Synthetic sequence
SLPLGLWAPNFN

SEQ ID NO: 23 - protein, 12 aa, Artificial Sequence
  Synthetic sequence
SLLIFRSWANFN

SEQ ID NO: 24 - protein, 12 aa, Artificial Sequence
  Synthetic sequence
SGVVIATVIVIT

SEQ ID NO: 25 - protein, 12 aa, Artificial Sequence
  Synthetic sequence
SLGPQGIWGQFN

SEQ ID NO: 26 - protein, 12 aa, Artificial Sequence
  Synthetic sequence
KKSPGRVVGGSV

SEQ ID NO: 27 - protein, 12 aa, Artificial Sequence
  Synthetic sequence
PQGLLGAPGILG

SEQ ID NO: 28 - protein, 31 aa, Artificial Sequence
  Synthetic sequence
HGPEGLRVGFYESDVMGRGHARLVHVEEPHT

SEQ ID NO: 29 - protein, 12 aa, Artificial Sequence
  Synthetic sequence
GPQGLAGQRGIV

SEQ ID NO: 30 - protein, 12 aa, Artificial Sequence
  Synthetic sequence
GGSGQRGRKALE

SEQ ID NO: 31 - protein, 12 aa, Artificial Sequence
  Synthetic sequence
SLSALLSSDIFN

SEQ ID NO: 32 - protein, 12 aa, Artificial Sequence
  Synthetic sequence
SLPRFKIIGGFN

SEQ ID NO: 33 - protein, 12 aa, Artificial Sequence
  Synthetic sequence
SLLGIAVPGNFN

SEQ ID NO: 34 - protein, 12 aa, Artificial Sequence
  Synthetic sequence
FFKNIVTPRTPP

SEQ ID NO: 35 - protein, 306 aa, Artificial Sequence
  Synthetic sequence
PPQIEEACELPECQVDAGNKVCNLQCNNHACGWDGGDCSLNFNDPWKNCTQSLQCWKYFSDGHCDSQCNSAGCLFDGFDCQLTEGQCNPLYDQYCKDHFSDGHCDQGCNSAECEWDGLDCAEHVPERLAAGTLVLVVLLPPDQLRNNSFHFLRELSHVLHTNVVFKRDAQGQQMIFPYYGHEEELRKHPIKRSTVGWATSSLLPGTSGGRQRRELDPMDIRGSIVYLEIDNRQCVQSSSQCFQSATDVAAFLGALASLGSLNIPYKIEAVKSEPVEPPLPSQLHLMYVAAAAFVLLFFVGCGVLLS

SEQ ID NO: 36 - protein, 358 aa, Artificial Sequence
  Synthetic sequence
PCVGSNPCYNQGTCEPTSENPFYRCLCPAKFNGLLCHILDYSFTGGAGRDIPPPQIEEACELPECQVDAGNKVCNLQCNNHACGWDGGDCSLNFNDPWKNCTQSLQCWKYFSDGHCDSQCNSAGCLFDGFDCQLTEGQCNPLYDQYCKDHFSDGHCDQGCNSAECEWDGLDCAEHVPERLAAGTLVLVVLLPPDQLRNNSFHFLRELSHVLHTNVVFKRDAQGQQMIFPYYGHEEELRKHPIKRSTVGWATSSLLPGTSGGRQRRELDPMDIRGSIVYLEIDNRQCVQSSSQCFQSATDVAAFLGALASLGSLNIPYKIEAVKSEPVEPPLPSQLHLMYVAAAAFVLLFFVGCGVLLS

SEQ ID NO: 37 - protein, 1114 aa, Bombyx mori
MMASTALSLMGRCNPDGCTRGKHVTAAPMDGPRGPSSLAGTFGWGLAIPAGEPCGRVCSPATVGFFPVAKKSNKENRPEASGLPLESERTGDNPTVRGSAGADPVGQDAPGWTCQFCERTFSTNRGLGVHKRRAHPVETNTDAAPMMVKRRWHGEEIDLLARTEARLLAERGQCSGGDLFGALPGFGRTLEAIKGQRRREPYRALVQAHLARFGSQPGPSSGGCSAEPDFRRASGAEEAGEERCAEDAAAYDPSAVGQMSPDAARVLSELLEGTGRRRACRAMRPKTAGRRNDLHDDRTASAHKTSRQKRRAVYARVQELYKKCRSRAAAEVIDGACGGVGHSLEEMETYWRPILERVSDAPGPTPEALHALGRAEWHGGNRDYTQLWKPISVEETKASRFDWRTSPGPYGIRSGQWRAVPVHLKAEMFNAWMARGEIPEVLRQCRTVFVPKVERPGGPGEYRPISIASIPLRHFHSILARRLLACCPPDARQRGFICADGTLENSAVLDAVLGDSRKKLWECHVAVLDFAKAFDTVSHEALVELLRLRGMPVQFCGYIAHLYDTASTTLAVNNEMSSPVKVGRGVRQGDPLSPILFNVVMDLILASLPERVGYRLEMEPVSALAYADDLVLLAGSKVGMQESISAVDCVGRQMGLRLNCRKSAVLSMIPGGHRKKHHYLTERTFNIGGKPLRQVSCVERWRYLGVDFEASGCVTLEHSISSALNNISRAPLKPQQRLEILRAHLIPRFQHGFVLGNISDDRLRMLDVQIRKAVGQWLRLPADVPKAYYHAAVQDGGLAIPSVRATIPDLIVRRFGGLDSSPWSVARAAAKSDKIRKKLRWAWKQLRRFSRVDSTTQRPSVRLFWREHLHASVDGRELRESTRTPTSTKWIRERCAQITGRDFVQFVHTHINALPSRIRGSRGRRGGGESSLTCRAGCKVRETTAHILQQCHRTRGGRILRHNKIVSFVAKAMEENKWTVELEPRLRTSVGLRKPDIIASRDGVGVIVDVQVVSGQRSLDELHREKRNKYGNHGELVELVAGRLGLPKAECVRATSCTISWRGVWSLTSYKELRSIIGLREPTLQIVPILALRGSHMNWTRFNQMTSVMGGGVG

SEQ ID NO: 38 - DNA, 1056 bp, Bombyx mori
ggccgcctcgtcaacgtgaagaaattcaagcaagcgcgggtaaacggcgggagtaactatgactctcttaagcgggagtaactatgactctcttaggggcgatacgcataattttaatttttcgattcaaatccagtcgtcttaatctggtgaccagtggcgcggtcaccagtatagtgcacaggacgtgaatggctccgaggctggcggagtcactcactataagtgtgagagacgatgtcctgtgccaagtatacgtccaaccctaacgggttaagtgaaattagttgctcataacagggacggtgtacctgtttgctcgtggctggctatcgaatggacgggaccaatacacccccctgttagtaatggggtaagagagagcggtctgaaactatggccgagatcacgacgccccactcctacccataacctgcacgtggtaccgccgcacattgaccgatacgggaggaggggcagcacttgaatcacgtagtcttggtgtagccattgcgggactacagccctcgtaagtgccgccttagaacgcaacggggcaataggtgggccggggcgctagcgggggggagtaatctcccctgttggcgtgcaccgcactgctccctctgggggcagtgtcatccggaaacaggtgggccggggcgccaccaggggggagcaatccctcctgatgatggcgagcaccgcactgtcccttatgggacggtgtaacccggatggctgtacacgtggtaaacacgtgacagcagccccgatggacggaccgcgaggaccgtcaagcctagcaggtaccttcgggtggggccttgcgatacctgcgggcgaaccctgtggtcgggtttgcagcccggccacagtgggtttttttcctgttgcaaaaaagtcaaataaagaaaatagacctgaagcctctggcctcccgctggagtcagagaggacaggcgataacccgactgtgcggggttccgccggcgcagatcctgtgggtcaggatgcgcctggttggacctgccagttctgcgaacgaaccttttc

SEQ ID NO: 39 - DNA, 502 bp, Bombyx mori
gagttaaggtccataatcgggcttcgggaaccgacactacaaatcgttccgatactggcgttgagaggttcacacatgaactggaccaggttcaatcagatgacgtccgtcatggggggcggcgttggttgagccttgcacagtagtccagcggtaagggtgtagatcaggcccgtctgtttctcccccggagctcgctcccttggcttcccttatatattttaacatcagaaacagacattaaacatctactgatccaatttcgccggcgtacggccacgatcgggagggtgggaatctcgggggtcttccgatcctaatccatgatgattacgacctgagtcactaaagacgatggcatgatgatccggcgatgaaaatagccaaatgcctcgtcatctaattagtgacgcgcatgaatggattaacgagattcccactgtccctatctactatctagcgaaaccacagccaagggaacgggcttgggagaatcagcggg

(19) United States
(12) Patent Application Publication
(10) Pub. No.: US 2021/0285009 A1
(43) Pub. Date: Sep. 16, 2021

(54) RETROTRANSPOSON-BASED DELIVERY VEHICLE AND METHODS OF USE THEREOF

(71) Applicant: The Regents of the University of California, Oakland, CA (US)

(72) Inventors: David V. Schaffer, Berkeley, CA (US); Christopher Barnes, Oakland, CA (US)

(21) Appl. No.: 17/257,796

(22) PCT Filed: Jul. 11, 2019
(86) PCT No.: PCT/US19/41472

§ 371 Date: Jan. 4, 2021

Related U.S. Application Data
(60) Provisional application No. 62/697,829, filed on Jul. 13, 2018.

(51) Int. Cl. C12N 15/85; C07K 14/725

(52) U.S. Cl. CPC C12N 15/85; C12N 2800/90; C07K 14/7051

(57) ABSTRACT

The present disclosure provides a gene delivery system comprising: a) an R2 retrotransposon R2 polypeptide, or a first nucleic acid comprising a nucleotide sequence encoding the R2 polypeptide; and b) a nucleic acid comprising a heterologous nucleotide sequence encoding one or more heterologous gene products, where the heterologous nucleotide sequence is flanked by an R2 retrotransposon 3′ untranslated region (UTR) and an R2 retrotransposon 5′ UTR, and where the heterologous nucleotide sequence has a length of at least 200 nucleotides. The present disclosure provides a method of delivering one or more gene products of interest to a eukaryotic cell, the method comprising contacting the cell with the gene delivery vehicle system.

### CROSS-REFERENCE

This application claims the benefit of U.S. Provisional Patent Application No. 62/697,829, filed Jul. 13, 2018, which application is incorporated herein by reference in its entirety.

### INTRODUCTION

Gene therapy is becoming an increasingly successful technology to treat human disease. Gene therapy involves the delivery of a nucleic acid that includes a coding region encoding a gene product of interest, where the gene product can provide a gain-of- or loss-of-functionality to correct aberrant behavior in specific cells. Delivery generally falls into two main categories: viral-mediated and non-viral mediated delivery. Additionally, delivery of a nucleic acid results in either transient expression or in non-reversible integration of all or a portion of the nucleic acid into the host cell's DNA. Viral-mediated integrative approaches are most commonly used in dividing cells, where delivery is mediated, e.g., through the use of lentiviruses and retroviruses engineered to carry therapeutic DNA into cells. Such viruses are effective at stable integration into the host cell's genome; however, they have a number of drawbacks. Integration into random locations in the cell's genome can result in disruption of the cell's function, and expression may even eventually be silenced by the cell own machinery. In addition, viral methods suffer severely in their capability to package and deliver a coding region encoding a gene product of interest, when the size of the coding region to be delivered exceeds 6 kilobases (kb) or 8 kb for retrovirus and lentivirus, respectively. While many gene therapies involve coding regions that are within the packaging limit, the length of the coding region for gene products already approaches the maximum limit at 8 kb, and the addition of larger cDNA, regulatory elements, or multiple genes would push the insert size to larger than can be accommodated by current viral delivery vehicles.

Class II transposons such as piggyBac, Sleeping Beauty, and Tol2 can integrate larger payloads; however, such transposons have drawbacks. For example, DNA transposons can integrate at specific but common sites in the genome (notably, transposable elements in general make up 45% of the genome), and some have a tendency to integrate in areas where active transcription is occurring.

There is a need in the art for delivery vehicles that provide for delivery of larger coding regions.

### SUMMARY

The present disclosure provides a gene delivery system comprising: a) a first nucleic acid comprising a nucleotide sequence encoding an R2 retrotransposon R2 polypeptide; and b) a second nucleic acid comprising a heterologous nucleotide sequence encoding one or more heterologous gene products, where the heterologous nucleotide sequence is flanked by an R2 retrotransposon 3′ untranslated region (UTR) and an R2 retrotransposon 5′ UTR, and where the heterologous nucleotide sequence has a length of up to about 15 kilobases. The present disclosure provides a method of delivering one or more gene products of interest to a eukaryotic cell, the method comprising contacting the cell with the gene delivery vehicle system.

### BRIEF DESCRIPTION OF THE DRAWINGS

FIG. 1A-1B depict: (FIG. 1A) Schematic of the dual vector integration system encoding the transgene flanked by the R2 UTR targeting sequences; and (FIG. 1B) Proposed mechanism of integration mechanism by R2.

FIG. 2 depicts analysis of the genomic integration junction in 28S rDNA of a gene delivered using a gene delivery system of the present disclosure.

FIG. 3 depicts analysis of the genomic integration junction in 28S rDNA of a gene delivered using a gene delivery system of the present disclosure.

FIG. 4 depicts a protocol for transfection and passaging of cells, and presents data showing green fluorescent protein (GFP) expression following transfection with a gene delivery system of the present disclosure.

FIGS. 5A and 5B depict the effect of optimized R2 (“OR2”) on transgene expression.

FIGS. 6A and 6B depict expression of a chimeric antigen receptor (CAR) following transfection of cells with a gene delivery system of the present disclosure, where the heterologous nucleic acid comprised a nucleotide sequence encoding the CAR.

FIG. 7 provides an amino acid sequence of an R2 polypeptide (SEQ ID NO:37).

FIG. 8 provides a nucleotide sequence of a 5′ UTR (SEQ ID NO:38).

FIG. 9 provides a nucleotide sequence of a 3′ UTR (SEQ ID NO:39).

FIG. 10 depicts the percent of HEK293 cells stably expressing green fluorescent protein (GFP) 14 days after transfection with a gene delivery system of the present disclosure.

FIG. 11 depicts the number of hygromycin-resistant HEK293 colonies 14 days after transfection with a gene delivery system of the present disclosure.

FIG. 12 depicts the percent of c-myc⁺ HEK293 cells 14 days after transfection with a gene delivery system of the present disclosure.

### DEFINITIONS

“Heterologous,” as used herein, means a nucleotide or polypeptide sequence that is not found in the native (e.g., naturally-occurring) nucleic acid or protein, respectively.

The terms “polynucleotide” and “nucleic acid,” used interchangeably herein, refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

“Operably linked” refers to a juxtaposition wherein the components so described are in a relationship permitting them to function in their intended manner. For instance, a promoter is operably linked to a coding sequence if the promoter affects its transcription or expression.

The terms “polypeptide,” “peptide,” and “protein”, used interchangeably herein, refer to a polymeric form of amino acids of any length, which can include genetically coded and non-genetically coded amino acids, chemically or biochemically modified or derivatized amino acids, and polypeptides having modified peptide backbones. The term includes fusion proteins, including, but not limited to, fusion proteins with a heterologous amino acid sequence, fusions with heterologous and homologous leader sequences, with or without N-terminal methionine residues; immunologically tagged proteins; and the like.

The terms “chimeric antigen receptor” and “CAR”, used interchangeably herein, refer to artificial multi-module molecules capable of triggering or inhibiting the activation of an immune cell which generally but not exclusively comprise an extracellular domain (e.g., a ligand/antigen binding domain), a transmembrane domain and one or more intracellular signaling domains. The term “CAR” is not limited specifically to CAR molecules but also includes CAR variants. CAR variants include split CARs wherein the extracellular portion (e.g., the ligand binding portion) and the intracellular portion (e.g., the intracellular signaling portion) of a CAR are present on two separate molecules. CAR variants also include ON-switch CARs which are conditionally activatable CARs, e.g., comprising a split CAR wherein conditional hetero-dimerization of the two portions of the split CAR is pharmacologically controlled. CAR variants also include bispecific CARs, which include a secondary CAR binding domain that can either amplify or inhibit the activity of a primary CAR. CAR variants also include inhibitory chimeric antigen receptors (iCARs) which may, e.g., be used as a component of a bispecific CAR system, where binding of a secondary CAR binding domain results in inhibition of primary CAR activation. CAR molecules and derivatives thereof (i.e., CAR variants) are described, e.g., in PCT Application No. US2014/016527; Fedorov et al. Sci Transl Med (2013); 5(215):215ra172; Glienke et al. Front Pharmacol (2015) 6:21; Kakarla & Gottschalk 52 Cancer J (2014) 20(2):151-5; Riddell et al. Cancer J (2014) 20(2):141-4; Pegram et al. Cancer J (2014) 20(2):127-33; Cheadle et al. Immunol Rev (2014) 257(1):91-106; Barrett et al. Annu Rev Med (2014) 65:333-47; Sadelain et al. Cancer Discov (2013) 3(4):388-98; Cartellieri et al., J Biomed Biotechnol (2010) 956304; the disclosures of which are incorporated herein by reference in their entirety.

“Single-chain Fv” or “sFv” antibody fragments comprise the V_{H} and V_{L} domains of antibody, wherein these domains are present in a single polypeptide chain. In some embodiments, the Fv polypeptide further comprises a polypeptide linker between the V_{H} and V_{L} domains, which enables the sFv to form the desired structure for antigen binding. For a review of sFv, see Pluckthun in The Pharmacology of Monoclonal Antibodies, vol. 113, Rosenburg and Moore eds., Springer-Verlag, New York, pp. 269-315 (1994).

The term “nanobody” (Nb), as used herein, refers to the smallest antigen binding fragment or single variable domain (V_{HH}) derived from naturally occurring heavy chain antibody and is known to the person skilled in the art. They are derived from heavy chain only antibodies, seen in camelids (Hamers-Casterman et al., 1993; Desmyter et al., 1996). In the family of “camelids” immunoglobulins devoid of light polypeptide chains are found. “Camelids” comprise old world camelids (Camelus bactrianus and Camelus dromedarius) and new world camelids (for example, Llama paccos, Llama glama, Llama guanicoe and Llama vicugna). A single variable domain heavy chain antibody is referred to herein as a nanobody or a V_{HH} antibody.

As used herein, the terms “treatment,” “treating,” “treat” and the like, refer to obtaining a desired pharmacologic and/or physiologic effect. The effect can be prophylactic in terms of completely or partially preventing a disease or symptom thereof and/or can be therapeutic in terms of a partial or complete cure for a disease and/or adverse effect attributable to the disease. “Treatment,” as used herein, covers any treatment of a disease in a mammal, particularly in a human, and includes: (a) preventing the disease from occurring in a subject which can be predisposed to the disease but has not yet been diagnosed as having it; (b) inhibiting the disease, i.e., arresting its development; and (c) relieving the disease, i.e., causing regression of the disease.

The terms “individual,” “subject,” “host,” and “patient,” used interchangeably herein, refer to a mammal, including, but not limited to, murines (rats, mice), non-human primates, humans, canines, felines, ungulates (e.g., equines, bovines, ovines, porcines, caprines), lagomorphs, etc. In some cases, the individual is a human. In some cases, the individual is a non-human primate. In some cases, the individual is a rodent, e.g., a rat or a mouse. In some cases, the individual is a lagomorph, e.g., a rabbit.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “an R2 polypeptide” includes a plurality of such polypeptides and reference to “the heterologous gene product” includes reference to one or more heterologous gene products and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION

The present disclosure provides a gene delivery system comprising: a) an R2 retrotransposon R2 polypeptide, or a first nucleic acid comprising a nucleotide sequence encoding the R2 polypeptide; and b) a nucleic acid comprising a heterologous nucleotide sequence encoding one or more heterologous gene products, where the heterologous nucleotide sequence is flanked by an R2 retrotransposon 3′ untranslated region (UTR) and an R2 retrotransposon 5′ UTR, and where the heterologous nucleotide sequence has a length of at least 200 nucleotides. The R2 polypeptide, the 5′UTR, and the 3′UTR provide for insertion of the heterologous nucleic acid into a 28S region of the genome of the eukaryotic cell. The present disclosure provides a method of delivering one or more gene products of interest to a eukaryotic cell, the method comprising contacting the cell with the gene delivery vehicle system.

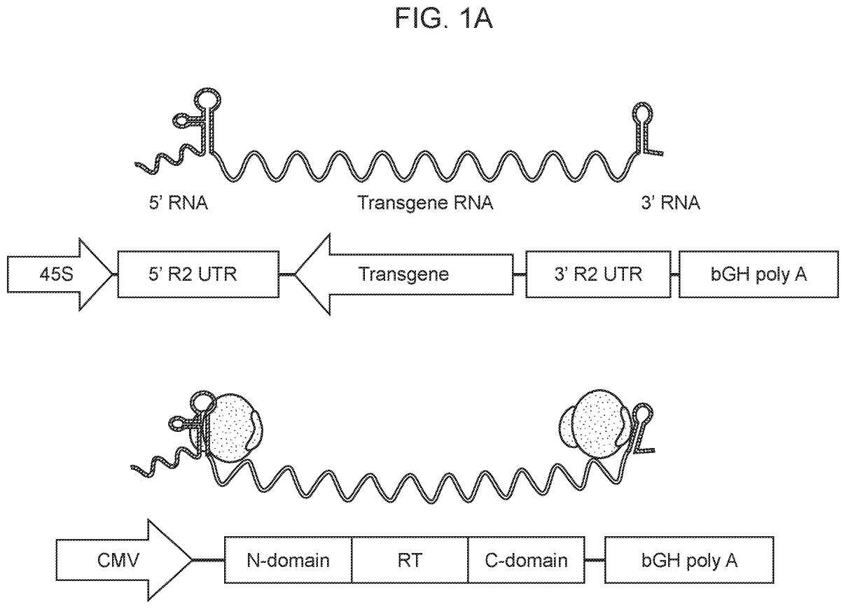

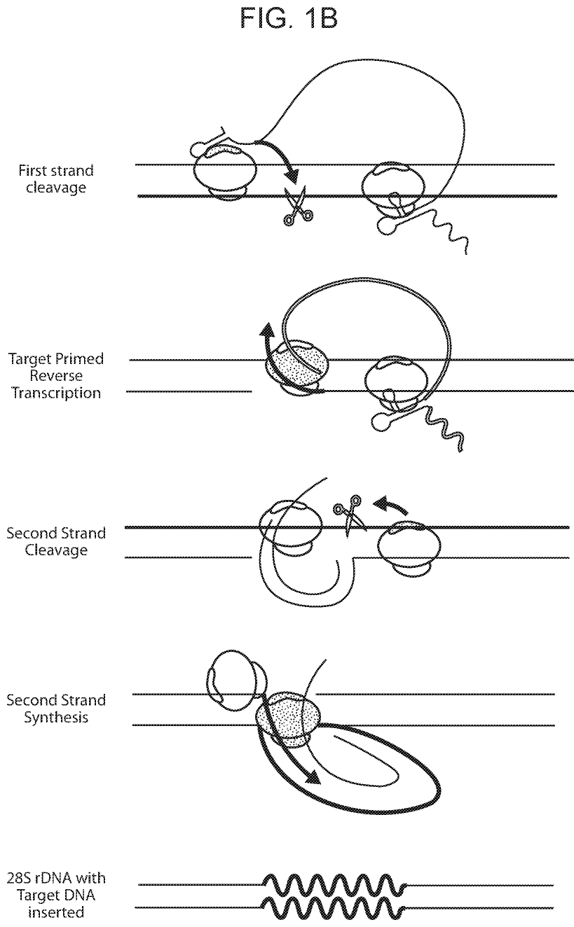

The R2 protein recognizes sites 5′ and 3′ of a DNA sequence that is conserved among a number of species in the 28S rDNA. The R2 protein interacts and binds transcribed RNA 5′ and 3′ of the R2 coding sequence. Based on whether the R2 protein binds the 5′ UTR or 3′ UTR, it will then interact with 28S DNA upstream or downstream of the target site. The current model of integration is that an R2 protein bound to 3′ UTR RNA will bind upstream of the cut site and nick the 3′ end via an endonuclease domain in the protein. From here, R2 will begin the process of Target-Primed Reverse Transcription (TPRT) and synthesize the 3′ strand of the DNA within the 28S region as proposed by Eickbush et al. (FIG. 1). Eickbush, et al. Microbiol. Spectr. 3, MDNA3-0011-2014 (2015).

Once that is completed, either the downstream associate R2 protein or native replication machinery begins second strand synthesis. The end result is integration of a transgene of interest (“heterologous nucleic acid” or “heterologous nucleotide sequence”) into the native 28S rDNA site.

Gene Delivery System

The present disclosure provides a gene delivery system comprising: a) a first nucleic acid comprising a nucleotide sequence encoding an R2 retrotransposon R2 polypeptide; and b) a second nucleic acid comprising a heterologous nucleotide sequence encoding one or more heterologous gene products, where the heterologous nucleotide sequence is flanked by an R2 retrotransposon 3′ UTR and an R2 retrotransposon 5′ UTR, and where the heterologous nucleotide sequence has a length of at least 200 nucleotides. The first and the second nucleic acids can be RNA. The first and the second nucleic acids can be DNA.

In some cases, the second nucleic acid comprises, in order from 5′ to 3′: i) an R2 5′UTR; ii) a promoter; iii) a heterologous nucleotide sequence encoding one or more heterologous gene products; and iv) an R2 3′UTR. In some cases, the second nucleic acid comprises, in order from 5′ to 3′: i) an R2 5′UTR; ii) a promoter; iii) a heterologous nucleotide sequence encoding one or more heterologous gene products; iv) a polyadenylation (polyA) sequence; and v) an R2 3′UTR. In some cases, e.g., where the promoter is an RNA polymerase II promoter, the heterologous nucleotide sequence encoding one or more heterologous gene products is in the opposite (reverse) orientation relative to the R2 5′UTR and the R2 3′UTR; i.e., the heterologous nucleotide sequence encoding one or more heterologous gene products is in the 3′-to-5′ orientation. In some cases, e.g., where the promoter is an RNA polymerase I promoter, the heterologous nucleotide sequence encoding one or more heterologous gene products is in the same orientation as the R2 5′UTR and the R2 3′UTR; i.e., the heterologous nucleotide sequence encoding one or more heterologous gene products is in the 5′-to-3′ orientation. The promoter is operably linked to the heterologous nucleotide sequence encoding one or more heterologous gene products. In some cases, the promoter is heterologous to the nucleotide sequence encoding one or more heterologous gene products.

The present disclosure provides a gene delivery system comprising: a) an R2 retrotransposon R2 polypeptide; and b) a nucleic acid comprising a heterologous nucleotide sequence encoding one or more heterologous gene products, where the heterologous nucleotide sequence is flanked by an R2 retrotransposon 3′ UTR and an R2 retrotransposon 5′ UTR, and where the heterologous nucleotide sequence has a length of at least 200 nucleotides.

R2 Polypeptides

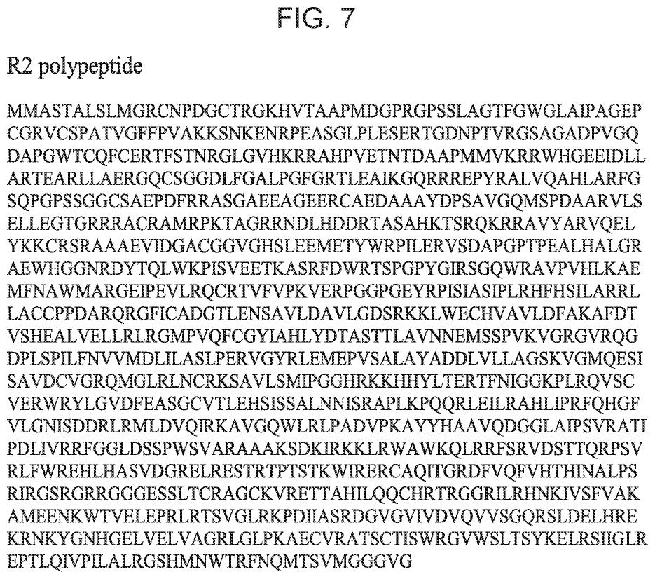

An R2 polypeptide encoded by the first nucleic acid of a gene delivery system of the present disclosure (where the gene delivery system comprises a first nucleic acid and a nucleic acid), or an R2 polypeptide present in a gene delivery system of the present disclosure (where the gene delivery system comprises an R2 polypeptide and a nucleic acid) can comprise an amino acid sequence having at least 85%, at least 90%, at least 95%, at least 98%, at least 99%, or 100%, amino acid sequence identity to the R2 amino acid sequence depicted in FIG. 7. The R2 polypeptide can have a length of from 1105 amino acids to 1125 amino acids, e.g., from about 1105 amino acids to about 1110 amino acids, from about 1110 amino acids to about 1115 amino acids, from about 1115 amino acids to about 1120 amino acids, or from about 1120 amino acids to about 1125 amino acids. In some cases, the R2 polypeptide has a length of 1114 amino acids. 5′UTR and 3′UTR

A suitable 5′UTR is any 5′UTR of an R2 retrotransposon. Nucleotide sequences of R2 retrotransposon 5′UTRs are known in the art; and any such 5′UTR can be included in a gene delivery system of the present disclosure.

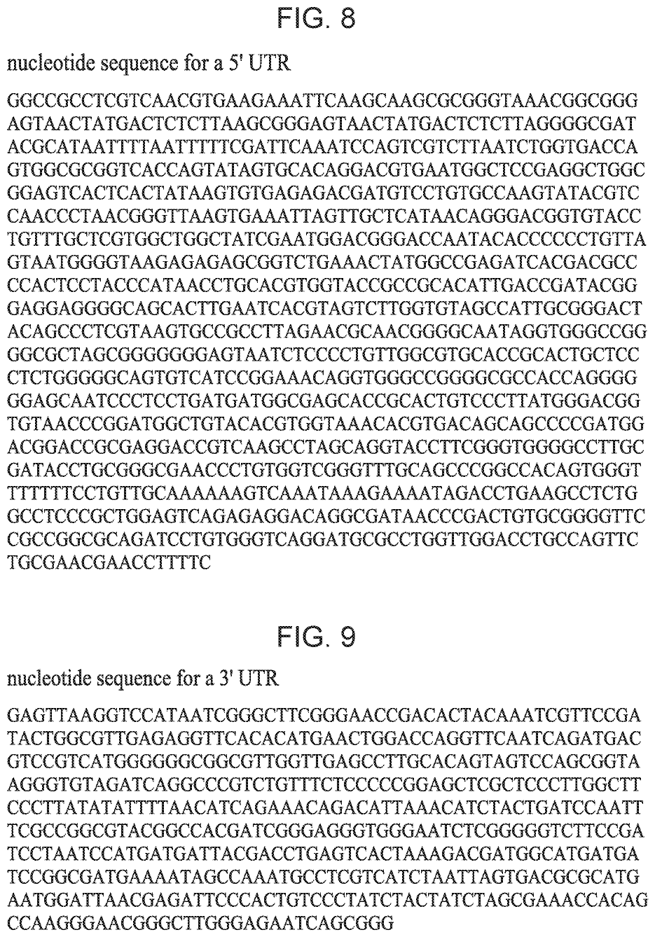

In some cases, a suitable 5′UTR comprises a nucleotide sequence having at least 85%, at least 90%, at least 95%, at least 98%, at least 99%, or 100%, nucleotide sequence identity to the nucleotide sequence depicted in FIG. 8. In some cases, a suitable 5′UTR has a length of from about 1000 nucleotides (nt) to about 1100 nt, e.g., from about 1000 nt to about 1025 nt, from about 1025 nt to about 1050 nt, from about 1050 nt to about 1075 nt, or from about 1075 nt to about 1100 nt. In some cases, a suitable 5′UTR has a length of from about 1050 nt to about 1060 nt. In some cases, a suitable 5′UTR has a length of 1056 nt.

In some cases, a suitable 3′UTR comprises a nucleotide sequence having at least 85%, at least 90%, at least 95%, at least 98%, at least 99%, or 100%, nucleotide sequence identity to the nucleotide sequence depicted in FIG. 9. In some cases, a suitable 3′UTR has a length of from about 475 nt to about 550 nt, e.g., from about 475 nt to about 500 nt, from about 500 nt to about 525 nt, or from about 525 nt to about 550 nt. In some cases, a suitable 3′UTR has a length of from about 500 nt to about 510 nt. In some cases, a suitable 3′UTR has a length of 502 nt.

Heterologous Nucleotide Sequence

As noted above, the second nucleic acid of a gene delivery system of the present disclosure comprises a heterologous nucleotide sequence (also referred to herein as “heterologous nucleic acid”) encoding one or more heterologous gene products, where the heterologous nucleotide sequence has a length of at least 200 nucleotides (nt). For example, in some cases, the heterologous nucleotide sequence has a length of from about 200 nt to about 300 nt, from about 300 nt to about 400 nt, from about 400 nt to about 500 nt, from about 500 nt to about 750 nt, from about 750 nt to about 1 kilobases (kb), from about 1 kb to about 1.5 kb, from about 1.5 kb to about 2 kb, from about 2 kb to about 2.5 kb, from about 2.5 kb to about 3 kb, or from about 3 kb to about 3.5 kb. As another example, in some cases, the heterologous nucleotide sequence has a length of from about 3.5 kb to about 4 kb, from about 4 kb to about 4.5 kb, from about 4.5 kb to about 5 kb, from about 5 kb to about 5.5 kb, from about 5.5 kb to about 6 kb, from about 6 kb to about 6.5 kb, from about 6.5 kb to about 7 kb, from about 7 kb to about 8 kb, from about 8 kb to about 9 kb, from about 9 kb to about 10 kb, from about 10 kb to about 11 kb, from about 11 kb to about 12 kb, from about 12 kb to about 13 kb, from about 13 kb to about 14 kb, or from about 14 kb to about 15 kb. In some cases, the heterologous nucleotide sequence has a length of from about 200 nt to about 1 kb. In some cases, the heterologous nucleotide sequence has a length of from about 1 kb to about 5 kb. In some cases, the heterologous nucleotide sequence has a length of from about 3.5 kb to about 6 kb. In some cases, the heterologous nucleotide sequence has a length of from about 6 kb to about 8 kb. In some cases, the heterologous nucleotide sequence has a length of from about 8 kb to about 15 kb. In some cases, the heterologous nucleotide sequence has a length of from about 9 kb to about 15 kb. In some cases, the heterologous nucleotide sequence has a length of from about 10 kb to about 15 kb.

In some cases, where the heterologous gene product is a polypeptide, the heterologous nucleotide sequence can encode a single heterologous gene product having a length of more than 50 amino acids. In some cases, where the heterologous gene product is a polypeptide, the heterologous nucleotide sequence can encode a single heterologous gene product having a length of more than 200 amino acids. In some cases, where the heterologous gene product is a polypeptide, the heterologous nucleotide sequence can encode a single heterologous gene product having a length of from about 50 amino acids (aa) to about 100 aa, from about 100 aa to about 200 aa, from about 200 aa to about 300 aa, from about 300 aa to about 400 aa, from about 400 aa to about 500 aa, from about 500 aa to about 750 aa, from about 750 aa to about 1000 aa, from about 1000 aa to about 1500 aa, from about 1500 aa to about 2000 aa, from about 2000 aa to about 2500 aa, or from about 2500 aa to about 3000 aa. In some cases, where the heterologous gene product is a polypeptide, the heterologous nucleotide sequence can encode a single heterologous gene product having a length of up to 3000 amino acids. In some cases, where the heterologous gene product is a polypeptide, the heterologous nucleotide sequence can encode a single heterologous gene product having a length of from about 3000 aa to about 5,000 aa. Where the heterologous gene products are two or more polypeptides, the heterologous nucleotide sequence can encode two or more heterologous gene products having a combined length of up to 3000 amino acids (aa), up to 4000 aa, or up to 5000 aa. Where the heterologous gene products are two or more polypeptides, the heterologous nucleotide sequence can encode two or more heterologous gene products having a combined length of more than 5,000 aa. Where the heterologous gene product is a nucleic acid, the heterologous nucleotide sequence can encode a single heterologous gene product having a length of at least 200 nt (e.g., from about 200 nt to about 500 nt, from about 500 nt to about 1 kb, from about 1 kb to about 3.5 kb, from about 3.5 kb to about 6 kb, from about 6 kb to about 10 kb, or from about 10 kb to about 15 kb). Where the heterologous gene products are two or more nucleic acids, the heterologous nucleotide sequence can encode heterologous gene products having a combined length of at least 200 nt (e.g., from about 200 nt to about 500 nt, from about 500 nt to about 1 kb, from about 1 kb to about 3.5 kb, from about 3.5 kb to about 6 kb, from about 6 kb to about 10 kb, or from about 10 kb to about 15 kb). Where a heterologous nucleotide sequence encodes a first gene product that is a nucleic acid and a second gene product that is a polypeptide, the heterologous nucleotide sequence can encode, e.g., any combination of lengths of the gene products such that the total combined length of the coding sequence encoding the two gene products is at least 200 nt (e.g., from about 200 nt to about 500 nt, from about 500 nt to about 1 kb, from about 1 kb to about 3.5 kb, from about 3.5 kb to about 6 kb, from about 6 kb to about 10 kb, or from about 10 kb to about 15 kb).

In some cases, the second nucleic acid of a gene delivery system of the present disclosure comprises a nucleotide sequence encoding a single heterologous polypeptide. In some cases, the second nucleic acid of a gene delivery system of the present disclosure comprises a nucleotide sequence encoding a single heterologous nucleic acid. In some cases, the second nucleic acid of a gene delivery system of the present disclosure comprises: a) a first nucleotide sequence encoding a first heterologous polypeptide; and b) a second nucleotide sequence encoding a second heterologous polypeptide. In some cases, the second nucleic acid of a gene delivery system of the present disclosure comprises: a) a first nucleotide sequence encoding a first heterologous polypeptide; b) a second nucleotide sequence encoding a second heterologous polypeptide; and c) a third nucleotide sequence encoding a third heterologous polypeptide. In some cases, the second nucleic acid of a gene delivery system of the present disclosure comprises: a) a first nucleotide sequence encoding a heterologous polypeptide; and b) a second nucleotide sequence encoding a heterologous nucleic acid. In some cases, the second nucleic acid of a gene delivery system of the present disclosure comprises: a) a first nucleotide sequence encoding a heterologous polypeptide; b) a second nucleotide sequence encoding a heterologous nucleic acid; and c) a third nucleotide sequence encoding a third heterologous nucleic acid.

Where the heterologous nucleotide sequence encodes two or more gene products (e.g., where the heterologous nucleotide sequence comprises a first nucleotide sequence encoding a first heterologous gene product and a second nucleotide sequence encoding a second heterologous gene product, etc.), in some cases, a nucleic acid linker is provided between the first nucleotide sequence and the second nucleotide sequence (or between any two nucleotide sequences encoding gene products). The nucleic acid linker can be an internal ribosomal entry site (IRES). The nucleic acid linker can comprise a nucleotide sequence encoding a self-cleaving 2A peptide (such as P2A, T2A, E2A, or F2A) linking the 3′ end of first nucleotide sequence to the 5′ end of the second nucleotide sequence.

In some cases, the second nucleic acid of a gene delivery system of the present disclosure comprises: a) a first nucleotide sequence encoding a first heterologous polypeptide; and b) a second nucleotide sequence encoding a second heterologous polypeptide; wherein the first and second nucleotide sequences are under the control of a single promoter. In some cases, the promoter is operably linked to the 5′ end of the first nucleotide sequence, and there is a nucleic acid linker selected from the group consisting of an IRES and a nucleic acid encoding a self-cleaving 2A peptide (such as P2A, T2A, E2A, or F2A) linking the 3′ end of first nucleotide sequence to the 5′ end of the second nucleotide sequence, where the first nucleotide sequence and the second nucleotide sequence are transcribed as a single RNA under the control of the promoter. In some cases, the promoter is operably linked to the 5′ end of the second nucleotide sequence, and there is nucleic acid linker selected from the group consisting of an IRES and a nucleic acid encoding a self-cleaving 2A peptide (such as P2A, T2A, E2A, or F2A) linking the 3′ end of second nucleotide sequence to the 5′ end of the first nucleotide sequence, where the first nucleotide sequence and the second nucleotide sequence are transcribed as a single RNA under the control of the promoter. In some cases, the promoter is inducible.

Self-cleaving viral 2A peptides suitable for use include the viral 2A peptide is a porcine teschovirus-1 (P2A), foot-and-mouth disease virus (F2A), Thosea asigna virus (T2A), equine rhinitis A virus (E2A), and viral porcine teschovirus-1 (P2A) peptide. P2A (GSGATNFSLLKQAGDVEENPGP (SEQ ID NO:1)), T2A (GSGEGRGSLLTCGDVEENPGP (SEQ ID NO:2)), E2A (GSGQCTNYALLKLAGDVESNPGP (SEQ ID NO:3)), and F2A (GSGVKQTLNFDLLKLAGDVESNPGP (SEQ ID NO:4)) can be considered as either “proteolytic cleavage sites” or “ribosome skipping signals” (CHYSEL). See, e.g., Kim et al. (2011) PLoS ONE 6:e18556. The mechanism by which the encoded polypeptides are generated as two polypeptide chains may be by self cleaving of the linker, by ribosome skipping, or translational shunting.

As an example, in some cases, the second nucleic acid of a gene delivery system of the present disclosure comprises: a) a first nucleotide sequence encoding a first heterologous polypeptide that is a first chain of a heterodimer; and b) a second nucleotide sequence encoding a second heterologous polypeptide that is the second chain of a heterodimer; wherein the first and second nucleotide sequences are under the control of a single promoter. In some cases, the second nucleic acid of a gene delivery system of the present disclosure comprises: a) a first nucleotide sequence encoding a first heterologous polypeptide that is a first chain of a heterodimer; b) a nucleic acid linker selected from the group consisting of an IRES and a nucleic acid encoding a self-cleaving 2A peptide; and c) a second nucleotide sequence encoding a second heterologous polypeptide that is the second chain of a heterodimer; wherein the first and second nucleotide sequences are under the control of a single promoter.

In some cases, the heterologous nucleotide sequence encodes a single polypeptide chain that is cleaved after translation to generate two polypeptide chains. For example, in some cases, the heterologous nucleotide sequence encodes a single polypeptide chain comprising, in order from N-terminus to C-terminus: i) a first polypeptide; ii) a proteolytically cleavable linker; and iii) a second polypeptide.

The proteolytically cleavable linker can include a protease recognition sequence recognized by a protease selected from the group consisting of alanine carboxypeptidase, Armillaria mellea astacin, bacterial leucyl aminopeptidase, cancer procoagulant, cathepsin B, clostripain, cytosol alanyl aminopeptidase, elastase, endoproteinase Arg-C, enterokinase, gastricsin, gelatinase, Gly-X carboxypeptidase, glycyl endopeptidase, human rhinovirus 3C protease, hypodermin C, IgA-specific serine endopeptidase, leucyl aminopeptidase, leucyl endopeptidase, lysC, lysosomal pro-X carboxypeptidase, lysyl aminopeptidase, methionyl aminopeptidase, myxobacter, nardilysin, pancreatic endopeptidase E, picornain 2A, picornain 3C, proendopeptidase, prolyl aminopeptidase, proprotein convertase I, proprotein convertase II, russellysin, saccharopepsin, semenogelase, T-plasminogen activator, thrombin, tissue kallikrein, tobacco etch virus (TEV), togavirin, tryptophanyl aminopeptidase, U-plasminogen activator, V8, venombin A, venombin AB, and Xaa-pro aminopeptidase.

For example, the proteolytically cleavable linker can comprise a matrix metalloproteinase cleavage site, e.g., a cleavage site for a MMP selected from collagenase-1, -2, and -3 (MMP-1, -8, and -13), gelatinase A and B (MMP-2 and -9), stromelysin 1, 2, and 3 (MMP-3, -10, and -11), matrilysin (MMP-7), and membrane metalloproteinases (MT1-MMP and MT2-MMP). For example, the cleavage sequence of MMP-9 is Pro-X-X-Hy (wherein, X represents an arbitrary residue; Hy, a hydrophobic residue; SEQ ID NO:5), e.g., Pro-X-X-Hy-(Ser/Thr) SEQ ID NO:6, e.g., Pro-Leu/Gln-Gly-Met-Thr-Ser (SEQ ID NO:7) or Pro-Leu/Gln-Gly-Met-Thr (SEQ ID NO:8). Another example of a protease cleavage site is a plasminogen activator cleavage site, e.g., a uPA or a tissue plasminogen activator (tPA) cleavage site. Another example of a suitable protease cleavage site is a prolactin cleavage site. Specific examples of cleavage sequences of uPA and tPA include sequences comprising Val-Gly-Arg. Another example of a protease cleavage site that can be included in a proteolytically cleavable linker is a tobacco etch virus (TEV) protease cleavage site, e.g., ENLYTQS (SEQ ID NO:9), where the protease cleaves between the glutamine and the serine. Another example of a protease cleavage site that can be included in a proteolytically cleavable linker is an enterokinase cleavage site, e.g., DDDDK (SEQ ID NO:10), where cleavage occurs after the lysine residue. Another example of a protease cleavage site that can be included in a proteolytically cleavable linker is a thrombin cleavage site, e.g., LVPR (SEQ ID NO:11). Additional suitable linkers comprising protease cleavage sites include linkers comprising one or more of the following amino acid sequences: LEVLFQGP (SEQ ID NO:12), cleaved by PreScission protease (a fusion protein comprising human rhinovirus 3C protease and glutathione-S-transferase; Walker et al. (1994) Biotechnol. 12:601); a thrombin cleavage site, e.g., CGLVPAGSGP (SEQ ID NO:13); SLLKSRMVPNFN (SEQ ID NO:14) or SLLIARRMPNFN (SEQ ID NO:15), cleaved by cathepsin B; SKLVQASASGVN (SEQ ID NO:16) or SSYLKASDAPDN (SEQ ID NO:17), cleaved by an Epstein-Barr virus protease; RPKPQQFFGLMN (SEQ ID NO:18) cleaved by MMP-3 (stromelysin); SLRPLALWRSFN (SEQ ID NO:19) cleaved by MMP-7 (matrilysin); SPQGIAGQRNFN (SEQ ID NO:20) cleaved by MMP-9; DVDERDVRGFASFL SEQ ID NO:21) cleaved by a thermolysin-like MMP; SLPLGLWAPNFN (SEQ ID NO:22) cleaved by matrix metalloproteinase 2(MMP-2); SLLIFRSWANFN (SEQ ID NO:23) cleaved by cathespin L; SGVVIATVIVIT (SEQ ID NO:24) cleaved by cathepsin D; SLGPQGIWGQFN (SEQ ID NO:25) cleaved by matrix metalloproteinase 1(MMP-1); KKSPGRVVGGSV (SEQ ID NO:26) cleaved by urokinase-type plasminogen activator; PQGLLGAPGILG (SEQ ID NO:27) cleaved by membrane type 1 matrixmetalloproteinase (MT-MMP); HGPEGLRVGFYESDVMGRGHARLVHVEEPHT (SEQ ID NO:28) cleaved by stromelysin 3 (or MMP-11), thermolysin, fibroblast collagenase and stromelysin-1; GPQGLAGQRGIV (SEQ ID NO:29) cleaved by matrix metalloproteinase 13 (collagenase-3); GGSGQRGRKALE (SEQ ID NO:30) cleaved by tissue-type plasminogen activator(tPA); SLSALLSSDIFN (SEQ ID NO:31) cleaved by human prostate-specific antigen; SLPRFKIIGGFN (SEQ ID NO:32) cleaved by kallikrein (hK3); SLLGIAVPGNFN (SEQ ID NO:33) cleaved by neutrophil elastase; and FFKNIVTPRTPP (SEQ ID NO:34) cleaved by calpain (calcium activated neutral protease).

In some cases, the second nucleic acid of a gene delivery system of the present disclosure comprises: a) a first nucleotide sequence encoding a first heterologous polypeptide; and b) a second nucleotide sequence encoding a second heterologous polypeptide; wherein the first and second nucleotide sequences are under the control of two different promoters. For example, in some cases, the second nucleic acid of a gene delivery system of the present disclosure comprises: a) a first nucleotide sequence encoding a first heterologous polypeptide, where the first nucleotide sequence is under control of a first promoter; and b) a second nucleotide sequence encoding a second heterologous polypeptide, where the second nucleotide sequence is under control of a second promoter. In some cases, the first promoter and the second promoter are both regulatable (e.g., inducible) promoters. In some cases, the first promoter and the second promoter are both constitutive promoters. In some cases, the first promoter is inducible, and the second promoter is constitutive. In some cases, the first promoter is constitutive, and the second promoter is inducible.

Transcriptional Control Elements

The heterologous nucleotide sequence present in the second nucleic acid of a gene delivery system of the present disclosure can be operably linked to a transcriptional control element(s). In some cases, the transcriptional control element is inducible. In some cases, the transcriptional control element is constitutive. In some cases, the transcriptional control element is a promoter. In some cases, the promoter is functional in a eukaryotic cell. In some cases, the promoter is a cell type-specific promoter. In some cases, the promoter is a tissue-specific promoter. In some cases, the promoter is constitutively active. In some cases, the promoter is a regulatable promoter.

A promoter can be a constitutively active promoter (i.e., a promoter that is constitutively in an active/“ON” state), it may be an inducible promoter (i.e., a promoter whose state, active/“ON” or inactive/“OFF”, is controlled by an external stimulus, e.g., the presence of a particular temperature, compound, or protein), it may be a spatially restricted promoter (i.e., transcriptional control element, enhancer, etc.)(e.g., tissue specific promoter, cell type specific promoter, etc.), and it may be a temporally restricted promoter (i.e., the promoter is in the “ON” state or “OFF” state during specific stages of embryonic development or during specific stages of a biological process, e.g., hair follicle cycle in mice).

Suitable promoter and enhancer elements are known in the art. For expression in a eukaryotic cell, suitable promoters include, but are not limited to, light and/or heavy chain immunoglobulin gene promoter and enhancer elements; cytomegalovirus immediate early promoter; herpes simplex virus thymidine kinase promoter; early and late SV40 promoters; promoter present in long terminal repeats from a retrovirus; mouse metallothionein-I promoter; and various art-known tissue specific promoters.

Suitable reversible promoters, including reversible inducible promoters are known in the art. Such reversible promoters may be isolated and derived from many organisms, e.g., eukaryotes and prokaryotes. Modification of reversible promoters derived from a first organism for use in a second organism, e.g., a first prokaryote and a second a eukaryote, a first eukaryote and a second a prokaryote, etc., is well known in the art. Such reversible promoters, and systems based on such reversible promoters but also comprising additional control proteins, include, but are not limited to, alcohol regulated promoters (e.g., alcohol dehydrogenase I (alcA) gene promoter, promoters responsive to alcohol transactivator proteins (AlcR), etc.), tetracycline regulated promoters, (e.g., promoter systems including TetActivators, TetON, TetOFF, etc.), steroid regulated promoters (e.g., rat glucocorticoid receptor promoter systems, human estrogen receptor promoter systems, retinoid promoter systems, thyroid promoter systems, ecdysone promoter systems, mifepristone promoter systems, etc.), metal regulated promoters (e.g., metallothionein promoter systems, etc.), pathogenesis-related regulated promoters (e.g., salicylic acid regulated promoters, ethylene regulated promoters, benzothiadiazole regulated promoters, etc.), temperature regulated promoters (e.g., heat shock inducible promoters (e.g., HSP-70, HSP-90, soybean heat shock promoter, etc.), light regulated promoters, synthetic inducible promoters, and the like. A suitable promoter can be an RNA pol I promoter. A suitable promoter can be an RNA pol II promoter.

Inducible promoters suitable for use include any inducible promoter described herein or known to one of ordinary skill in the art. Examples of inducible promoters include, without limitation, chemically/biochemically-regulated and physically-regulated promoters such as alcohol-regulated promoters, tetracycline-regulated promoters (e.g., anhydrotetracycline (aTc)-responsive promoters and other tetracycline-responsive promoter systems, which include a tetracycline repressor protein (tetR), a tetracycline operator sequence (tetO) and a tetracycline transactivator fusion protein (tTA)), steroid-regulated promoters (e.g., promoters based on the rat glucocorticoid receptor, human estrogen receptor, moth ecdysone receptors, and promoters from the steroid/retinoid/thyroid receptor superfamily), metal-regulated promoters (e.g., promoters derived from metallothionein (proteins that bind and sequester metal ions) genes from yeast, mouse and human), pathogenesis-regulated promoters (e.g., induced by salicylic acid, ethylene or benzothiadiazole (BTH)), temperature/heat-inducible promoters (e.g., heat shock promoters), and light-regulated promoters (e.g., light responsive promoters from plant cells).

In some cases, the promoter is a CD8 cell-specific promoter, a CD4 cell-specific promoter, a neutrophil-specific promoter, or an NK-specific promoter. For example, a CD4 gene promoter can be used; see, e.g., Salmon et al. (1993) Proc. Natl. Acad. Sci. USA 90: 7739; and Marodon et al. (2003) Blood 101:3416. As another example, a CD8 gene promoter can be used. NK cell-specific expression can be achieved by use of an Ncrl (p46) promoter; see, e.g., Eckelhart et al. (2011) Blood 117:1565.

In some cases, the promoter is a cardiomyocyte-specific promoter. In some cases, the promoter is a smooth muscle cell-specific promoter. In some cases, the promoter is a neuron-specific promoter. In some cases, the promoter is an adipocyte-specific promoter. Other cell type-specific promoters are known in the art and are suitable for use herein.

Heterologous Gene Products

Any of a variety of heterologous gene products can be encoded by a heterologous nucleotide sequence present in the second nucleic acid of a gene delivery system of the present disclosure. The heterologous gene product can be a single heterologous polypeptide. The heterologous gene product can be a single nucleic acid. The heterologous gene products can be two or more heterologous polypeptides. The heterologous gene products can be two or more heterologous nucleic acids. The heterologous gene products can be: i) a heterologous polypeptide; and ii) a heterologous nucleic acid.

Where the heterologous gene product is a polypeptide, suitable polypeptides include, but are not limited to, receptors, enzymes, antibodies, homodimeric polypeptides, heterodimeric polypeptides, polypeptide hormones, extracellular matrix proteins, proteoglycans, nucleases, RNA-guided CRISPR/Cas effector polypeptides, chimeric polypeptides, fusion polypeptides, and the like. In some cases, the heterologous gene product is a CAR. In some cases, the heterologous gene product is a synNotch polypeptide. In some cases, the heterologous gene product is a synNotch polypeptide and a CAR.

Where the heterologous gene product is a nucleic acid, suitable nucleic acids include, but are not limited to, an RNA that comprises a nucleotide sequence that encodes a polypeptide; a micro RNA; a ribozyme; an inhibitory RNA; a guide RNA that comprises a first segment that is complementary to a nucleotide sequence in a target nucleic acid and a second segment that binds to an RNA-guided effector polypeptide, and the like. In some cases, the heterologous gene product is a short interfering RNA (siRNA), a short hairpin RNA (shRNA), a ribozyme, a microRNA (miRNA), a small temporal RNA (stRNA), an antisense RNA, a small RNA-induced gene activation (RNAa), or a small activating RNA (saRNA).

Chimeric Antigen Receptor

As one non-limiting example, a heterologous polypeptide is a chimeric antigen receptor (CAR). In some cases, the CAR is a single polypeptide chain CAR. In other cases, the CAR is a heterodimeric CAR comprising two polypeptide chains. A single polypeptide chain CAR can comprise: i) an antigen binding domain; ii) a transmembrane domain; and iii) an intracellular signalling domain. A single polypeptide chain CAR can comprise: i) an antigen binding domain; ii) a transmembrane domain; iii) an immunomodulatory domain; and iv) an intracellular signalling domain.

The antigen-binding portion of a CAR can be an antibody or an antibody fragment. “Antibody fragments” comprise a portion of an intact antibody, for example, the antigen binding or variable region of the intact antibody. Examples of antibody fragments include Fab, Fab′, F(ab′)₂, and Fv fragments; single-chain Fv (scFv); diabodies; linear antibodies (Zapata et al., Protein Eng. 8(10): 1057-1062 (1995)); chimeric antibodies; humanized antibodies; single-chain antibodies (scAb); single domain antibodies (dAb); single domain heavy chain antibodies; single domain light chain antibodies; nanobodies; bi-specific antibodies; multi-specific antibodies; and fusion proteins comprising an antigen-binding (also referred to herein as antigen binding) portion of an antibody and a non-antibody protein. In some cases, the antigen-binding domain is a scFv. In some cases, the antigen-binding domain is a nanobody. Other antibody based antigen-binding domains (cAb VHH (camelid antibody variable domains) and humanized versions, IgNAR VH (shark antibody variable domains) and humanized versions, sdAb VH (single domain antibody variable domains) and “camelized” antibody variable domains are suitable for use.

The antigen-binding domain of a CAR can have a variety of antigen-binding specificities. In some cases, the antigen-binding domain is specific for an epitope present in an antigen that is expressed by (synthesized by) a cancer cell, i.e., a cancer cell associated antigen. The cancer cell associated antigen can be an antigen associated with, e.g., a breast cancer cell, a B cell lymphoma, a Hodgkin lymphoma cell, an ovarian cancer cell, a prostate cancer cell, a mesothelioma, a lung cancer cell (e.g., a small cell lung cancer cell), a non-Hodgkin B-cell lymphoma (B-NHL) cell, an ovarian cancer cell, a prostate cancer cell, a mesothelioma cell, a lung cancer cell (e.g., a small cell lung cancer cell), a melanoma cell, a chronic lymphocytic leukemia cell, an acute lymphocytic leukemia cell, a neuroblastoma cell, a glioma, a glioblastoma, a medulloblastoma, a colorectal cancer cell, etc. A cancer cell associated antigen may also be expressed by a non-cancerous cell.

Non-limiting examples of antigens to which an antigen-binding domain of a CAR can bind include, e.g., CD19, CD20, CD38, CD30, Her2/neu, ERBB2, CA125, MUC-1, prostate-specific membrane antigen (PSMA), CD44 surface adhesion molecule, mesothelin, carcinoembryonic antigen (CEA), epidermal growth factor receptor (EGFR), EGFRvIII, vascular endothelial growth factor receptor-2 (VEGFR2), high molecular weight-melanoma associated antigen (HMW-MAA), MAGE-A1, IL-13R-a2, GD2, and the like.

Suitable immunomodulatory domains (also referred to as “costimulatory domains” or “costimulatory polypeptides”) include, e.g., 4-1BB (CD137), CD28, ICOS, OX-40, BTLA, CD27, CD30, GITR, and HVEM.

Suitable intracellular signalling domains include, e.g., polypeptides that include one or more immunoreceptor tyrosine-based activation motifs (ITAMs). An ITAM motif is YX₁X₂L/I, where X₁ and X2 are independently any amino acid. Examples of suitable ITAM motif-containing polypeptides include, but are not limited to: DAP12; FCER1G (Fc epsilon receptor I gamma chain); CD3D (CD3 delta); CD3E (CD3 epsilon); CD3G (CD3 gamma); CD3Z (CD3 zeta); and CD79A (antigen receptor complex-associated protein alpha chain).

In some cases, a CAR is a heterodimeric CAR comprising two polypeptide chains. See, e.g., U.S. Pat. Nos. 9,821,012 and 9,587,020. For example, in some cases, a heterodimeric CAR comprises: a) a first polypeptide chain comprising: i) an extracellular antigen binding domain that specifically binds an antigen on a target cell; ii) a transmembrane domain; and iii) a first member of a dimerization pair; and b) a second polypeptide comprising: i) a transmembrane domain; ii) a second member of the dimerization pair; and iii) an intracellular signaling domain comprising an ITAM, where the intracellular signaling domain provides signal transduction activity. The first polypeptide, the second polypeptide or both the first and second polypeptides of the heterodimeric CAR comprise a costimulatory polypeptide. When present in a eukaryotic cell membrane, the first polypeptide of the CAR binds an antigen and the CAR dimerizes in the presence of a small molecule dimerizer.

In some cases, a heterodimeric CAR comprises: a) a first polypeptide comprising, in order from N-terminus to C-terminus: i) an antigen binding domain (e.g., an antigen-binding single-chain Fv (scFv) or a nanobody); ii) a transmembrane domain; and iii) a first member of a dimerization pair; and b) a second polypeptide comprising, in order from N-terminus to C-terminus: i) a transmembrane domain; ii) a second member of the dimerization pair; and iii) an intracellular signaling domain comprising an ITAM, wherein the intracellular signaling domain provides signal transduction activity, where the first polypeptide, the second polypeptide or both the first and second polypeptides comprise a costimulatory polypeptide interposed between the transmembrane domain and the member of the dimerization pair. In some cases, the costimulatory polypeptide is a 4-1BB polypeptide. In some cases, the costimulatory polypeptide is a CD28 polypeptide. In some cases, the costimulatory polypeptide is an OX-40 polypeptide. In some cases, the first polypeptide comprises a hinge region between the antigen-binding domain (e.g., scFv, nanobody, etc.) and the transmembrane domain. In some cases, the intracellular signaling domain comprising the ITAM is selected from the group consisting of CD3-zeta and ZAP70.

Suitable dimerization pairs include, e.g., a) FK506 binding protein (FKBP) and FKBP; b) FKBP and calcineurin catalytic subunit A (CnA); c) FKBP and cyclophilin; d) FKBP and FKBP-rapamycin associated protein (FRB); e) gyrase B (GyrB) and GyrB; f) dihydrofolate reductase (DHFR) and DHFR; g) DmrB and DmrB; h) PYL and ABI; i) Cry2 and CIB1; and j) GAI and GID1.

Suitable dimerizers (“dimerizing agents) that can provide for dimerization of a first member of a dimerizer-binding pair and a second member of a dimerizer-binding pair include, e.g. (where the dimerizer is in parentheses following the dimerizer-binding pair: a) FKBP and FKBP (rapamycin); b) FKBP and CnA (rapamycin); c) FKBP and cyclophilin (rapamycin); d) FKBP and FRG (rapamycin); e) GyrB and GyrB (coumermycin); f) DHFR and DHFR (methotrexate); g) DmrB and DmrB (AP20187); h) PYL and ABI (abscisic acid); i) Cry2 and CIB1 (blue light); and j) GAI and GID1 (gibberellin).

synNotch

As another non-limiting example, a heterologous nucleotide sequence can encode a synNotch polypeptide (also referred to herein as a “chimeric Notch receptor polypeptide.” Suitable synNotch polypeptides are described in, e.g., U.S. Pat. No. 9,670,281; Morsut et al. (2016) Cell 164:780; and Roybal et al. (2016) Cell 167:419. A synNotch polypeptide comprises: i) an antigen-binding domain; ii) a portion of a Notch polypeptide; and iii) an intracellular domain (e.g., a transcription factor (e.g., a transcriptional activator or a transcriptional repressor), a site-specific nuclease, etc.). A synNotch polypeptide does not bind Delta, the naturally-occurring ligand of Notch. Instead, a synNotch polypeptide binds an antigen that is bound by the antigen-binding domain present in the synNotch polypeptide. Binding of the antigen-binding domain to an antigen (e.g., an antigen present on a cell, such as a cancer cell) induces cleavage at an S2 proteolytic cleavage site and/or an S3 proteolytic cleavage site in the Notch polypeptide, thereby releasing the intracellular domain

In some cases, a synNotch polypeptide comprises: i) an antigen-binding domain; ii) a Notch regulatory region comprising a Lin 12-Notch repeat, a heterodimerization domain comprising an S2 proteolytic cleavage site and a transmembrane domain comprising an S3 proteolytic cleavage site; and iii) an intracellular domain, heterologous to the Notch regulatory region, comprising a transcriptional activator comprising a DNA binding domain, where binding of the antigen-binding domain to an antigen in trans induces cleavage at the S2 and S3 proteolytic cleavage sites, thereby releasing the intracellular domain

In some cases, a synNotch polypeptide comprises: i) an antigen-binding domain; ii) a Notch regulatory region comprising a Lin 12-Notch repeat, an S2 proteolytic cleavage site, and a transmembrane domain comprising an S3 proteolytic cleavage site; and iii) an intracellular domain comprising a transcriptional activator or a transcriptional repressor that is heterologous to the Notch regulatory region, where binding of the first member of the specific binding pair to the second member of the specific binding pair, present on a cell or other solid support, induces cleavage at the S2 and S3 proteolytic cleavage sites, thereby releasing the intracellular domain

The antigen-binding portion of a synNotch polypeptide can be an antibody or an antibody fragment. “Antibody fragments” comprise a portion of an intact antibody, for example, the antigen binding or variable region of the intact antibody. Examples of antibody fragments include Fab, Fab′, F(ab′)₂, and Fv fragments; single-chain Fv (scFv); diabodies; linear antibodies (Zapata et al., Protein Eng. 8(10): 1057-1062 (1995)); chimeric antibodies; humanized antibodies; single-chain antibodies (scAb); single domain antibodies (dAb); single domain heavy chain antibodies; single domain light chain antibodies; nanobodies; bi-specific antibodies; multi-specific antibodies; and fusion proteins comprising an antigen-binding (also referred to herein as antigen binding) portion of an antibody and a non-antibody protein. In some cases, the antigen-binding domain is a scFv. In some cases, the antigen-binding domain is a nanobody. Other antibody based antigen-binding domains (cAb VHH (camelid antibody variable domains) and humanized versions, IgNAR VH (shark antibody variable domains) and humanized versions, sdAb VH (single domain antibody variable domains) and “camelized” antibody variable domains are suitable for use.

The antigen-binding domain of a synNotch polypeptide can have a variety of antigen-binding specificities. In some cases, the antigen-binding domain is specific for an epitope present in an antigen that is expressed by (synthesized by) a cancer cell, i.e., a cancer cell associated antigen. The cancer cell associated antigen can be an antigen associated with, e.g., a breast cancer cell, a B cell lymphoma, a Hodgkin lymphoma cell, an ovarian cancer cell, a prostate cancer cell, a mesothelioma, a lung cancer cell (e.g., a small cell lung cancer cell), a non-Hodgkin B-cell lymphoma (B-NHL) cell, an ovarian cancer cell, a prostate cancer cell, a mesothelioma cell, a lung cancer cell (e.g., a small cell lung cancer cell), a melanoma cell, a chronic lymphocytic leukemia cell, an acute lymphocytic leukemia cell, a neuroblastoma cell, a glioma, a glioblastoma, a medulloblastoma, a colorectal cancer cell, etc. A cancer cell associated antigen may also be expressed by a non-cancerous cell.

Non-limiting examples of antigens to which an antigen-binding domain of a synNotch polypeptide can bind include, e.g., CD19, CD20, CD38, CD30, Her2/neu, ERBB2, CA125, MUC-1, prostate-specific membrane antigen (PSMA), CD44 surface adhesion molecule, mesothelin, carcinoembryonic antigen (CEA), epidermal growth factor receptor (EGFR), EGFRvIII, vascular endothelial growth factor receptor-2 (VEGFR2), high molecular weight-melanoma associated antigen (HMW-MAA), MAGE-A1, IL-13R-a2, GD2, and the like.

In in some cases, the antigen-binding domain of a synNotch polypeptide is an scFv. As another example, in some cases, the antigen-binding domain of a synNotch polypeptide is a nanobody.

In some cases, the Notch polypeptide present in a synNotch polypeptide comprises an amino acid sequence having at least 75%, at least 80%, at least 85%, at least 90%, at least 95%, at least 98%, or 100% amino acid sequence identity to the following sequence: PPQIEEACELPECQVDAGNKVCNLQCNNHACGWDGGDCSLNFNDPWKNCTQSLQCWK YFSDGHCDSQCNSAGCLFDGFDCQLTEGQCNPLYDQYCKDHFSDGHCDQGCNSAECE WDGLDCAEHVPERLAAGTLVLVVLLPPDQLRNNSFHFLRELSHVLHTNVVFKRDAQGQ QMIFPYYGHEEELRKHPIKRSTVGWATSSLLPGTSGGRQRRELDPMDIRGSIVYLEIDNR QCVQSSSQCFQSATDVAAFLGALASLGSLNIPYKIEAVKSEPVEPPLPSQLHLMYVAAAA FVLLFFVGCGVLLS (SEQ ID NO:35). In some cases, the Notch polypeptide present in a synNotch polypeptide comprises an amino acid sequence having at least 75%, at least 80%, at least 85%, at least 90%, at least 95%, at least 98%, or 100% amino acid sequence identity to the following sequence:

(SEQ ID NO: 36)
PCVGSNPCYNQGTCEPTSENPFYRCLCPAKFNGLLCHILDYSFTGGAGR
DIPPPQIEEACELPECQVDAGNKVCNLQCNNHACGWDGGDCSLNFNDPW
KNCTQSLQCWKYFSDGHCDSQCNSAGCLFDGFDCQLTEGQCNPLYDQYC
KDHFSDGHCDQGCNSAECEWDGLDCAEHVPERLAAGTLVLVVLLPPDQL
RNNSFHFLRELSHVLHTNVVFKRDAQGQQMIFPYYGHEEELRKHPIKRS
TVGWATSSLLPGTSGGRQRRELDPMDIRGSIVYLEIDNRQCVQSSSQCF
QSATDVAAFLGALASLGSLNIPYKIEAVKSEPVEPPLPSQLHLMYVAAA
AFVLLFFVGCGVLLS.

In some cases, the intracellular domain is a transcription factor. Suitable transcription factors include, e.g., ASCL1, BRN2, CDX2, CDX4, CTNNB1, EOMES, JUN, FOS, HNF4a, HOXAs (e.g., HOXA1, HOXA2, HOXA3, HOXA4, HOXA5, HOXA10, HOXA11, HOXA13), HOXBs (e.g., HOXB9), HOXCs (e.g., HOXC4, HOXCS, HOXC6, HOXC8, HOXC9, HOXC10, HOXC11, HOXC12, HOXC13), HOXDs (e.g., HOXD1, HOXD3, HOXD4, HOXD8, HOXD9, HOXD10, HOXD11, HOXD12, HOXD13), SNAI1-3, MYOD1, MYOG, NEUROD1-6 (e.g., NEUROD1, NEUROD2, NEUROD4, NEUROD6), PDX1, PU.1, SOX2, Nanog, Klf4, BCL-6, SOX9, STAT1-6, TBET, TCF, TEAD1-4 (e.g., TEAD1, TEAD2, TEAD3, TEAD4), TAF6L, CLOCK, CREB, GATA3, IRF7, MycC, NFkB, RORyt, RUNX1, SRF, TBX21, NFAT, MEF2D, and FoxP3.

In some cases, the intracellular domain is a transcription factor having a regulatory role in one or more immune cells (i.e., an immune cell regulatory transcription factor). Suitable immune cell regulatory transcription factors include, e.g., 2210012G02Rik, Akap8l, Appl2, Arid4b, Arid5b, Ash11, Atf7, Atm, C430014K11Rik, Chd9, Dmtf1, Fos, Foxo1, Foxp1, Hmbox1, Kdm5b, Klf2, Mga, Mll1, Mll3, Myst4, Pcgf6, Rev3l, Scml4, Scp2, Smarca2, Ssbp2, Suhw4, Tcf7, Tfdp2, Tox, Zbtb20, Zbtb44, Zeb1, Zfml, Zfp1, Zfp319, Zfp329, Zfp35, Zfp386, Zfp445, Zfp518, Zfp652, Zfp827, Zhx2, Eomes, Arntl, Bbx, Hbp1, Jun, Mef2d, Mterfd1, Nfat5, Nfe212, Nr1d2, Phf21a, Taf4b, Trf, Zbtb25, Zfp326, Zfp451, Zfp58, Zfp672, Egr2, Ikzf2, Taf1d, Chrac1, Dnajb6, Aplp2, Batf, Bhlhe40, Fosb, Hist1h1c, Hopx, Ifih1, Ikzf3, Lass4, Lin54, Mxd1, Mxi1, Prdm1, Prf1, Rora, Rpa2, Sap30, Stat2, Stat3, Taf9b, Tbx21, Trps1, Xbp1, Zeb2, Atf3, Cenpc1, Lass6, Rb1, Zbtb41, Crem, Fosl2, Gtf2b, Irf7, Maff, Nr4a1, Nr4a2, Nr4a3, Obfc2a, Rbl2, Rel, Rybp, Sra1, Tgif1, Tnfaip3, Uhrf2, Zbtb1, Ccdc124, Csda, E2f3, Epas1, H1f0, H2afz, Hif1a, Ikzf5, Irf4, Nsbp1, Pim1, Rfc2, Swap70, Tfb1m, 2610036L11Rik, 5133400G04Rik, Apitd1, Blm, Brca1, Brip1, C1d, C79407, Cenpa, Cfl1, Clspn, Ddx1, Dscc1, E2f7, E2f8, Ercc6l, Ezh2, Fen1, Foxm1, Gen1, Gsg2, H2afx, Hdac1, Hdgf, Hells, Hist1h1e, Hist3h2a, Hjurp, Hmgb2, Hmgb3, Irf1, Irf8, Kif22, Kif4, Lig1, Lmo2, Lnp, Mbd4, Mcm2, Mcm3, Mcm4, Mcm5, Mcm6, Mcm7, Mybl2, Neil3, Nusap1, Orc6l, Pola1, Pola2, Pole, Pole2, Polh, Polr2f, Polr2j, Ppp1r8, Prim2, Psmc3ip, Rad51, Rad51c, Rad54l, Rfc3, Rfc4, Rnps1, Rpa1, Smarcc1, Spic, Ssrp1, Taf9, Tfdp1, Tmpo, Topbp1, Trdmt1, Uhrf1, Wdhd1, Whsc1, Zbp1, Zbtb32, Zfp367, Car1, Polg2, Atr, Lef1, Myc, Nucb2, Satb1, Taf1a, Ift57, Apex1, Chd7, Chtf8, Ctnnb1, Etv3, Irf9, Myb, Mybbp1a, Pms2, Preb, Sp110, Stat1, Trp53, Zfp414, App, Cdk9, Ddb1, Hsf2, Lbr, Pa2g4, Rbms1, Rfc1, Rfc5, Tada2l, Tex261, Xrcc6, and the like.

RNA-Guided Effector Polypeptides and Guide RNAs

As another non-limiting example, a heterologous nucleotide sequence can encode an RNA-guided effector polypeptide. As another non-limiting example, a heterologous nucleotide sequence can comprise: i) a first nucleotide sequence encoding an RNA-guided effector polypeptide; and ii) a second nucleotide sequence encoding a guide RNA. As another non-limiting example, a heterologous nucleotide sequence can comprise: i) a first nucleotide sequence encoding an RNA-guided effector polypeptide; ii) a second nucleotide sequence encoding a first guide RNA; and iii) a third nucleotide sequence encoding a second guide RNA.

Suitable RNA-guided effector polypeptides include, e.g., CRISPR/Cas endonucleases (e.g., class 2 CRISPR/Cas endonucleases such as a type II, type V, or type VI CRISPR/Cas endonucleases). A suitable RNA-guided effector polypeptide is a CRISPR/Cas endonuclease (e.g., a class 2 CRISPR/Cas endonuclease such as a type II, type V, or type VI CRISPR/Cas endonuclease). In some cases, a RNA-guided effector polypeptide is a class 2 CRISPR/Cas endonuclease. In some cases, a suitable RNA-guided effector polypeptide is a class 2 type II CRISPR/Cas endonuclease (e.g., a Cas9 protein). In some cases, a suitable RNA-guided effector polypeptide is a class 2 type V CRISPR/Cas endonuclease (e.g., a Cpf1 protein, a C2c1 protein, or a C2c3 protein). In some cases, a suitable RNA-guided effector polypeptide is a class 2 type VI CRISPR/Cas endonuclease (e.g., a C2c2 protein; also referred to as a “Cas13a” protein). Also suitable for use is a CasX protein. Also suitable for use is a CasY protein. Also suitable for use are RNA-guided effector polypeptides that have decreased nuclease activity but retain target nucleic acid binding activity when complexed with a guide RNA. Also suitable for use are RNA-guided effector polypeptides that have substantially no nuclease activity but retain target nucleic acid binding activity when complexed with a guide RNA. Also suitable for use are RNA-guided effector polypeptides that exhibit nickase activity. Also suitable for use are RNA-guided effector polypeptides that cleave RNA.

Examples of various Cas9 proteins (and Cas9 domain structure) and Cas9 guide RNAs (as well as information regarding requirements related to protospacer adjacent motif (PAM) sequences present in targeted nucleic acids) can be found in the art, for example, see Jinek et al., Science. 2012 Aug. 17; 337(6096):816-21; Chylinski et al., RNA Biol. 2013 May; 10(5):726-37; Ma et al., Biomed Res Int. 2013; 2013:270805; Hou et al., Proc Natl Acad Sci USA. 2013 Sep. 24; 110(39):15644-9; Jinek et al., Elife. 2013; 2:e00471; Pattanayak et al., Nat Biotechnol. 2013 September; 31(9):839-43; Qi et al., Cell. 2013 Feb. 28; 152(5):1173-83; Wang et al., Cell. 2013 May 9; 153(4):910-8; Auer et al., Genome Res. 2013 Oct. 31; Chen et al., Nucleic Acids Res. 2013 Nov. 1; 41(20):e19; Cheng et al., Cell Res. 2013 October; 23(10):1163-71; Cho et al., Genetics. 2013 November; 195(3):1177-80; DiCarlo et al., Nucleic Acids Res. 2013 April; 41(7):4336-43; Dickinson et al., Nat Methods. 2013 October; 10(10):1028-34; Ebina et al., Sci Rep. 2013; 3:2510; Fujii et al., Nucleic Acids Res. 2013 Nov. 1; 41(20):e187; Hu et al., Cell Res. 2013 November; 23(11):1322-5; Jiang et al., Nucleic Acids Res. 2013 Nov. 1; 41(20):e188; Larson et al., Nat Protoc. 2013 November; 8(11):2180-96; Mali et al., Nat Methods. 2013 October; 10(10):957-63; Nakayama et al., Genesis. 2013 December; 51(12):835-43; Ran et al., Nat Protoc. 2013 November; 8(11):2281-308; Ran et al., Cell. 2013 Sep. 12; 154(6):1380-9; Upadhyay et al., G3 (Bethesda). 2013 Dec. 9; 3(12):2233-8; Walsh et al., Proc Natl Acad Sci USA. 2013 Sep. 24; 110(39):15514-5; Xie et al., Mol Plant. 2013 Oct. 9; Yang et al., Cell. 2013 Sep. 12; 154(6):1370-9; Briner et al., Mol Cell. 2014 Oct. 23; 56(2):333-9; Shmakov et al., Nat Rev Microbiol. 2017 March; 15(3):169-182; and U.S. patents and patent applications: U.S. Pat. Nos. 8,906,616; 8,895,308; 8,889,418; 8,889,356; 8,871,445; 8,865,406; 8,795,965; 8,771,945; 8,697,359; 20140068797; 20140170753; 20140179006; 20140179770; 20140186843; 20140186919; 20140186958; 20140189896; 20140227787; 20140234972; 20140242664; 20140242699; 20140242700; 20140242702; 20140248702; 20140256046; 20140273037; 20140273226; 20140273230; 20140273231; 20140273232; 20140273233; 20140273234; 20140273235; 20140287938; 20140295556; 20140295557; 20140298547; 20140304853; 20140309487; 20140310828; 20140310830; 20140315985; 20140335063; 20140335620; 20140342456; 20140342457; 20140342458; 20140349400; 20140349405; 20140356867; 20140356956; 20140356958; 20140356959; 20140357523; 20140357530; 20140364333; and 20140377868; each of which is hereby incorporated by reference in its entirety.

In some cases, a suitable RNA-guided effector polypeptide is a variant Cas9 protein. A variant Cas9 protein has an amino acid sequence that is different by at least one amino acid (e.g., has a deletion, insertion, substitution, fusion) when compared to the amino acid sequence of a corresponding wild type Cas9 protein. In some instances, the variant Cas9 protein has an amino acid change (e.g., deletion, insertion, or substitution) that reduces the nuclease activity of the Cas9 protein. For example, in some instances, the variant Cas9 protein has 50% or less, 40% or less, 30% or less, 20% or less, 10% or less, 5% or less, or 1% or less of the nuclease activity of the corresponding wild-type Cas9 protein. In some cases, the variant Cas9 protein has no substantial nuclease activity. When a Cas9 protein is a variant Cas9 protein that has no substantial nuclease activity, it can be referred to as a nuclease defective Cas9 protein or “dCas9” for “dead” Cas9. A protein (e.g., a class 2 CRISPR/Cas protein, e.g., a Cas9 protein) that cleaves one strand but not the other of a double stranded target nucleic acid is referred to herein as a “nickase” (e.g., a “nickase Cas9”).

Also suitable for use are fusion RNA-guided effector polypeptides, where a fusion RNA-guided effector polypeptide includes: a) an RNA-guided effector polypeptide; and b) a heterologous fusion partner. In some cases the fusion partner has enzymatic activity that modifies the target nucleic acid (e.g., ssRNA, dsRNA, ssDNA, dsDNA). Examples of enzymatic activity that can be provided by the fusion partner include but are not limited to: nuclease activity such as that provided by a restriction enzyme (e.g., FokI nuclease), methyltransferase activity such as that provided by a methyltransferase (e.g., HhaI DNA m5c-methyltransferase (M.HhaI), DNA methyltransferase 1 (DNMT1), DNA methyltransferase 3a (DNMT3a), DNA methyltransferase 3b (DNMT3b), METI, DRM3 (plants), ZMET2, CMT1, CMT2 (plants), and the like); demethylase activity such as that provided by a demethylase (e.g., Ten-Eleven Translocation (TET) dioxygenase 1 (TET1CD), TET1, DME, DML1, DML2, ROS1, and the like), DNA repair activity, DNA damage activity, deamination activity such as that provided by a deaminase (e.g., a cytosine deaminase enzyme such as rat APOBEC1), dismutase activity, alkylation activity, depurination activity, oxidation activity, pyrimidine dimer forming activity, integrase activity such as that provided by an integrase and/or resolvase (e.g., Gin invertase such as the hyperactive mutant of the Gin invertase, GinH106Y; human immunodeficiency virus type 1 integrase (IN); Tn3 resolvase; and the like), transposase activity, recombinase activity such as that provided by a recombinase (e.g., catalytic domain of Gin recombinase), polymerase activity, ligase activity, helicase activity, photolyase activity, and glycosylase activity). In some cases, the fusion partner is a nuclease, e.g., a FokI nuclease. In some cases, the heterologous fusion partner is a deaminase. Suitable deaminases include a cytidine deaminase and an adenosine deaminase.

In some cases, an RNA-guided effector polypeptide, or a fusion RNA-guided effector polypeptide, comprises one or more nuclear localization signals (NLSs). In some cases, an RNA-guided effector polypeptide, or a fusion RNA-guided effector polypeptide, comprises a cell penetrating peptide. In some cases, an RNA-guided effector polypeptide, or a fusion RNA-guided effector polypeptide, comprises an endosmolytic peptide.

In some cases, a guide RNA includes two separate nucleic acid molecules: an “activator” and a “targeter” and is referred to herein as a “dual guide RNA”, a “double-molecule guide RNA”, a “two-molecule guide RNA”, or a “dgRNA.” In some cases, the guide RNA is one molecule (e.g., for some class 2 CRISPR/Cas proteins, the corresponding guide RNA is a single molecule; and in some cases, an activator and targeter are covalently linked to one another, e.g., via intervening nucleotides), and the guide RNA is referred to as a “single guide RNA”, a “single-molecule guide RNA,” a “one-molecule guide RNA”, or simply “sgRNA.”

Compositions

The present disclosure provides a composition comprising a gene delivery system of the present disclosure.

A composition of the present disclosure comprises: a) a gene delivery system of the present disclosure; and b) at least one additional component, where suitable additional components include, e.g., a salt, a buffer, a protease inhibitor, a nuclease inhibitor, a lipid, and the like. In some cases, a composition of the present disclosure comprises: a) a gene delivery system of the present disclosure; and b) a lipid. In some cases, a composition of the present disclosure comprises: a) a gene delivery system of the present disclosure; and b) a liposome, a hydrogel, a microparticle, a nanoparticle, or a block copolymer micelle.

A composition of the present disclosure can include: a) a gene delivery system of the present disclosure; and b) one or more of: a buffer, a surfactant, an antioxidant, a hydrophilic polymer, a dextrin, a chelating agent, a suspending agent, a solubilizer, a thickening agent, a stabilizer, a bacteriostatic agent, a wetting agent, and a preservative. Suitable buffers include, but are not limited to, (such as N,N-bis(2-hydroxyethyl)-2-aminoethanesulfonic acid (BES), bis(2-hydroxyethyl)amino-tris(hydroxymethyl)methane (BIS-Tris), N-(2-hydroxyethyl)piperazine-N′3-propanesulfonic acid (EPPS or HEPPS), glycylglycine, N-2-hydroxyehtylpiperazine-N′-2-ethanesulfonic acid (HEPES), 3-(N-morpholino)propane sulfonic acid (MOPS), piperazine-N,N′-bis(2-ethane-sulfonic acid) (PIPES), sodium bicarbonate, 3-(N-tris(hydroxymethyl)-methyl-amino)-2-hydroxy-propanesulfonic acid) TAPSO, (N-tris(hydroxymethyl)methyl-2-aminoethanesulfonic acid (TES), N-tris(hydroxymethyl)methyl-glycine (Tricine), tris(hydroxymethyl)-aminomethane (Tris), etc.). Suitable salts include, e.g., NaCl, MgCl₂, KCl, MgSO₄, etc.

A composition of the present disclosure can include: a) a gene delivery system of the present disclosure; and b) a pharmaceutically acceptable excipient. Pharmaceutically acceptable excipients have been amply described in a variety of publications, including, for example, A. Gennaro (2000) “Remington: The Science and Practice of Pharmacy”, 20th edition, Lippincott, Williams, & Wilkins; Pharmaceutical Dosage Forms and Drug Delivery Systems (1999) H. C. Ansel et al., eds 7^{th} ed., Lippincott, Williams, & Wilkins; and Handbook of Pharmaceutical Excipients (2000) A. H. Kibbe et al., eds., 3^{rd} ed. Amer. Pharmaceutical Assoc.

In some cases, a gene delivery system of the present disclosure is in a particle, or is associated with a particle. The terms “particle” and “nanoparticle” can be used interchangeable, as appropriate.

A gene delivery system of the present disclosure can be present in or associated with a particle, e.g., a delivery particle comprising lipid or lipidoid and hydrophilic polymer, e.g., a cationic lipid and a hydrophilic polymer, for instance wherein the cationic lipid comprises 1,2-dioleoyl-3-trimethylammonium-propane (DOTAP) or 1,2-ditetradecanoyl-sn-glycero-3-phosphocholine (DMPC) and/or wherein the hydrophilic polymer comprises ethylene glycol or polyethylene glycol (PEG); and/or wherein the particle further comprises cholesterol (e.g., particle from formulation 1=DOTAP 100, DMPC 0, PEG 0, Cholesterol 0; formulation number 2=DOTAP 90, DMPC 0, PEG 10, Cholesterol 0; formulation number 3=DOTAP 90, DMPC 0, PEG 5, Cholesterol 5). For example, a particle can be formed using a multistep process in which a gene delivery system of the present disclosure is mixed together, e.g., at a 1:1 molar ratio, e.g., at room temperature, e.g., for 30 minutes, e.g., in sterile, nuclease free 1× phosphate-buffered saline (PBS); and separately, DOTAP, DMPC, PEG, and cholesterol as applicable for the formulation are dissolved in alcohol, e.g., 100% ethanol; and, the two solutions are mixed together to form particles containing the gene delivery system of the present disclosure).

A gene delivery system of the present disclosure can be part of a nanoparticle. For example, a biodegradable core-shell structured nanoparticle with a poly (β-amino ester) (PBAE) core enveloped by a phospholipid bilayer shell can be used. In some cases, particles/nanoparticles based on self assembling bioadhesive polymers are used; such particles/nanoparticles may be applied to oral delivery, intravenous delivery, and nasal delivery.

In some cases, a composition of the present disclosure comprises a gene delivery system of the present disclosure and poly(beta-amino alcohol) (PBAA). US Patent Publication No. 20130302401 relates to a class of poly(beta-amino alcohols) (PBAAs) that has been prepared using combinatorial polymerization.

In some cases, a composition of the present disclosure comprises a gene delivery system of the present disclosure and one or more lipid nanoparticles (LNPs). Negatively charged polymers such as RNA may be loaded into LNPs at low pH values (e.g., pH 4) where the ionizable lipids display a positive charge. However, at physiological pH values, the LNPs exhibit a low surface charge compatible with longer circulation times. Four species of ionizable cationic lipids have been focused upon, namely 1,2-dilineoyl-3-dimethylammonium-propane (DLinDAP), 1,2-dilinoleyloxy-3-N,N-dimethylaminopropane (DLinDMA), 1,2-dilinoleyloxy-keto-N,N-dimethyl-3-aminopropane (DLinKDMA), and 1,2-dilinoleyl-4-(2-dimethylaminoethyl)-[1,3]-dioxolane (DLinKC2-DMA). Preparation of LNPs and is described in, e.g., Rosin et al. (2011) Molecular Therapy 19:1286-2200). The cationic lipids 1,2-dilineoyl-3-dimethylammonium-propane (DLinDAP), 1,2-dilinoleyloxy-3-N,N-dimethylaminopropane (DLinDMA), 1,2-dilinoleyloxyketo-N,N-dimethyl-3-aminopropane (DLinK-DMA), 1,2-dilinoleyl-4-(2-dimethylaminoethyl)-[1,3]-dioxolane (DLinKC2-DMA), (3-o[2″-(methoxypolyethyleneglycol 2000) succinoyl]-1,2-dimyristoyl-sn-glycol (PEG-S-DMG), and R-3-[(omega-methoxy-poly(ethylene glycol)2000) carbamoyl]-1,2-dimyristyloxlpropyl-3-amine (PEG-C-DOMG) may be used. A nucleic acid (e.g., a guide RNA; a nucleic acid of the present disclosure; etc.) may be encapsulated in LNPs containing DLinDAP, DLinDMA, DLinK-DMA, and DLinKC2-DMA (cationic lipid:DSPC:CHOL: PEGS-DMG or PEG-C-DOMG at 40:10:40:10 molar ratios). In some cases, 0.2% SP-DiOC18 is used.

In some cases, a composition of the present disclosure comprises a gene delivery system of the present disclosure and spherical Nucleic Acid (SNA™) constructs or other nanoparticles (particularly gold nanoparticles). See, e.g., Cutler et al., J. Am. Chem. Soc. 2011 133:9254-9257, Hao et al., Small 2011 7:3158-3162, Zhang et al., ACS Nano. 2011 5:6962-6970, Cutler et al., J. Am. Chem. Soc. 2012 134:1376-1391, Young et al., Nano Lett. 2012 12:3867-71, Zheng et al., Proc. Natl. Acad. Sci. USA. 2012 109:11975-80, Mirkin, Nanomedicine 2012 7:635-638 Zhang et al., J. Am. Chem. Soc. 2012 134:16488-1691, Weintraub, Nature 2013 495:S14-S16, Choi et al., Proc. Natl. Acad. Sci. USA. 2013 110(19): 7625-7630, Jensen et al., Sci. Transl. Med. 5, 209ra152 (2013) and Mirkin, et al., Small, 10:186-192.

In some cases, a gene delivery system of the present disclosure is present in, or associated with, a nanoparticle. In general, a “nanoparticle” refers to any particle having a diameter of less than 1000 nm. In some cases, nanoparticles suitable for use in delivering a gene delivery system of the present disclosure to a target cell have a diameter of 500 nm or less, e.g., from 25 nm to 35 nm, from 35 nm to 50 nm, from 50 nm to 75 nm, from 75 nm to 100 nm, from 100 nm to 150 nm, from 150 nm to 200 nm, from 200 nm to 300 nm, from 300 nm to 400 nm, or from 400 nm to 500 nm. In some cases, nanoparticles suitable for use in delivering a gene delivery system of the present disclosure to a target cell have a diameter of from 25 nm to 200 nm. In some cases, nanoparticles suitable for use in delivering a gene delivery system of the present disclosure to a target cell have a diameter of 100 nm or less. In some cases, nanoparticles suitable for use in delivering a gene delivery system of the present disclosure to a target cell have a diameter of from 35 nm to 60 nm.

Nanoparticles may be provided in various forms, e.g., as solid nanoparticles (e.g., metal such as silver, gold, iron, titanium), non-metal, lipid-based solids, polymers), suspensions of nanoparticles, or combinations thereof. Metal, dielectric, and semiconductor nanoparticles may be prepared, as well as hybrid structures (e.g., core-shell nanoparticles). Nanoparticles made of semiconducting material may also be labeled quantum dots if they are small enough (typically below 10 nm) that quantization of electronic energy levels occurs. Such nanoscale particles are used in biomedical applications as drug carriers or imaging agents and may be adapted for similar purposes in the present disclosure.

Semi-solid and soft nanoparticles are also suitable for inclusion in a composition of the present disclosure comprising a gene delivery system of the present disclosure. A prototype nanoparticle of semi-solid nature is the liposome.

In some cases, a composition of the present disclosure comprises a gene delivery system of the present disclosure and a liposome. Liposomes are spherical vesicle structures composed of a uni- or multilamellar lipid bilayer surrounding internal aqueous compartments and a relatively impermeable outer lipophilic phospholipid bilayer. Liposomes can be made from several different types of lipids; however, phospholipids are most commonly used to generate liposomes. Although liposome formation is spontaneous when a lipid film is mixed with an aqueous solution, it can also be expedited by applying force in the form of shaking by using a homogenizer, sonicator, or an extrusion apparatus. Several other additives may be added to liposomes in order to modify their structure and properties. For instance, either cholesterol or sphingomyelin may be added to the liposomal mixture in order to help stabilize the liposomal structure and to prevent the leakage of the liposomal inner cargo. A liposome formulation may be mainly comprised of natural phospholipids and lipids such as 1,2-distearoryl-sn-glycero-3-phosphatidyl choline (DSPC), sphingomyelin, egg phosphatidylcholines and monosialoganglioside.

In some cases, a composition of the present disclosure comprises a gene delivery system of the present disclosure and a stable nucleic-acid-lipid particle (SNALP). The SNALP formulation may contain the lipids 3-N-[(methoxypoly(ethylene glycol) 2000) carbamoyl]-1,2-dimyristyloxy-propylamine (PEG-C-DMA), 1,2-dilinoleyloxy-N,N-dimethyl-3-aminopropane (DLinDMA), 1,2-distearoyl-sn-glycero-3-phosphocholine (DSPC) and cholesterol, in a 2:40:10:48 molar percent ratio. The SNALP liposomes can be about 80-100 nm in size. A SNALP may comprise synthetic cholesterol (Sigma-Aldrich, St Louis, Mo., USA), dipalmitoylphosphatidylcholine (Avanti Polar Lipids, Alabaster, Ala., USA), 3-N-[(w-methoxy poly(ethylene glycol)2000)carbamoyl]-1,2-dimyrestyloxypropylamine, and cationic 1,2-dilinoleyloxy-3-N,Ndimethylaminopropane. A SNALP may comprise synthetic cholesterol (Sigma-Aldrich), 1,2-distearoyl-sn-glycero-3-phosphocholine (DSPC; Avanti Polar Lipids Inc.), PEG-cDMA, and 1,2-dilinoleyloxy-3-(N;N-dimethyl)aminopropane (DLinDMA).

Other cationic lipids, such as amino lipid 2,2-dilinoleyl-4-dimethylaminoethyl-[1,3]-dioxolane (DLin-KC2-DMA) can be used included in a composition of the present disclosure. A preformed vesicle with the following lipid composition may be contemplated: amino lipid, distearoylphosphatidylcholine (DSPC), cholesterol and (R)-2,3-bis(octadecyloxy) propyl-1-(methoxy poly(ethylene glycol)2000)propylcarbamate (PEG-lipid) in the molar ratio 40/10/40/10, respectively, and a FVII siRNA/total lipid ratio of approximately 0.05 (w/w). To ensure a narrow particle size distribution in the range of 70-90 nm and a low polydispersity index of 0.11.+−.0.04 (n=56), the particles may be extruded up to three times through 80 nm membranes prior to adding a gene delivery system. Particles containing the highly potent amino lipid 16 may be used, in which the molar ratio of the four lipid components 16, DSPC, cholesterol and PEG-lipid (50/10/38.5/1.5) which may be further optimized to enhance in vivo activity.

Lipids may be formulated with a system of the present disclosure or component(s) thereof or nucleic acids encoding the same to form lipid nanoparticles (LNPs). Suitable lipids include, but are not limited to, DLin-KC2-DMA4, C12-200 and colipids disteroylphosphatidyl choline, cholesterol, and PEG-DMG may be formulated with a system, or component thereof, of the present disclosure, using a spontaneous vesicle formation procedure. The component molar ratio may be about 50/10/38.5/1.5 (DLin-KC2-DMA or C12-200/disteroylphosphatidyl choline/cholesterol/PEG-DMG).

A gene delivery system of the present disclosure may be encapsulated in PLGA microspheres such as that further described in US published applications 20130252281 and 20130245107 and 20130244279.

In some cases, a composition of the present disclosure comprises a gene delivery system of the present disclosure and a supercharged protein. Supercharged proteins are a class of engineered or naturally occurring proteins with unusually high positive or negative net theoretical charge. Both super-negatively and super-positively charged proteins exhibit the ability to withstand thermally or chemically induced aggregation. Super-positively charged proteins are also able to penetrate mammalian cells. Associating cargo with these proteins, such as plasmid DNA, RNA, or other proteins, can facilitate the functional delivery of these macromolecules into mammalian cells both in vitro and in vivo.

The present disclosure also provides an implantable device comprising a gene delivery system of the present disclosure. The implantable device can include a container (e.g., a reservoir, a matrix, etc.) comprising a gene delivery system of the present disclosure, e.g., comprising a composition comprising a gene delivery system of the present disclosure. A suitable implantable device can comprise a polymeric substrate, such as a matrix for example, that is used as the device body, and in some cases additional scaffolding materials, such as metals or additional polymers, and materials to enhance visibility and imaging. An implantable delivery device can be advantageous in providing release locally and over a prolonged period, where the nucleic acid to be delivered is released directly to a target site, e.g., the extracellular matrix (ECM), the vasculature surrounding a tumor, a diseased tissue, etc.

Suitable implantable delivery devices include devices suitable for use in delivering to a cavity such as the abdominal cavity and/or any other type of administration in which the delivery system is not anchored or attached, comprising a biostable and/or degradable and/or bioabsorbable polymeric substrate, which may for example optionally be a matrix. In some cases, a suitable implantable delivery device comprises degradable polymers, wherein the main release mechanism is bulk erosion. In some cases, a suitable implantable delivery device comprises non degradable, or slowly degraded polymers, wherein the main release mechanism is diffusion rather than bulk erosion, so that the outer part functions as membrane, and its internal part functions as a reservoir, which practically is not affected by the surroundings for an extended period (for example from about a week to about a few months). Combinations of different polymers with different release mechanisms may also optionally be used.

In some cases, the implantable delivery system is designed to shield the nucleotide based therapeutic agent (gene delivery system of the present disclosure) from degradation, whether chemical in nature or due to attack from enzymes and other factors in the body of the subject.

### Kits

The present disclosure provides a kit comprising: a) a first nucleic acid comprising a nucleotide sequence encoding an R2 retrotransposon R2 polypeptide; and b) a second nucleic acid comprising an insertion site for inserting a heterologous nucleotide sequence encoding one or more heterologous gene products, wherein the insertion site is flanked by an R2 retrotransposon 3′ untranslated region (UTR) and an R2 retrotransposon 5′ UTR, and wherein the heterologous nucleotide sequence has a length of at least 200 nucleotides. The second nucleic acid thus allows for insertion of a heterologous nucleotide sequence encoding any desired heterologous gene product(s). In some cases, the first nucleic acid and the second nucleic acid are in separate containers. In some cases, the second nucleic acid further comprises a transcriptional control element 5′ of the insertion site. The transcriptional control element is positioned relative to the insertion site such that, once a heterologous nucleotide sequence is inserted into the second nucleic acid, the transcriptional control element is operably linked to the heterologous nucleotide sequence. Suitable transcriptional control elements are as described above.

The present disclosure provides a kit comprising: a) an R2 retrotransposon R2 polypeptide; and b) a nucleic acid comprising an insertion site for inserting a heterologous nucleotide sequence encoding one or more heterologous gene products, wherein the insertion site is flanked by an R2 retrotransposon 3′ UTR and an R2 retrotransposon 5′ UTR, and wherein the heterologous nucleotide sequence has a length of at least 200 nucleotides. The nucleic acid thus allows for insertion of a heterologous nucleotide sequence encoding any desired heterologous gene product(s). In some cases, the R2 polypeptide and the nucleic acid are in separate containers. In some cases, the nucleic acid further comprises a transcriptional control element 5′ of the insertion site. The transcriptional control element is positioned relative to the insertion site such that, once a heterologous nucleotide sequence is inserted into the nucleic acid, the transcriptional control element is operably linked to the heterologous nucleotide sequence. Suitable transcriptional control elements are as described above.

As noted above, the nucleic acid that allows for insertion of a heterologous nucleotide sequence encoding any desired heterologous gene product(s) provides for insertion of a heterologous nucleotide sequence of at least 200 nucleotides (nt). For example, in some cases, the heterologous nucleotide sequence has a length of from about 200 nt to about 300 nt, from about 300 nt to about 400 nt, from about 400 nt to about 500 nt, from about 500 nt to about 750 nt, from about 750 nt to about 1 kilobases (kb), from about 1 kb to about 1.5 kb, from about 1.5 kb to about 2 kb, from about 2 kb to about 2.5 kb, from about 2.5 kb to about 3 kb, or from about 3 kb to about 3.5 kb. As another example, in some cases, the heterologous nucleotide sequence has a length of from about 3.5 kb to about 4 kb, from about 4 kb to about 4.5 kb, from about 4.5 kb to about 5 kb, from about 5 kb to about 5.5 kb, from about 5.5 kb to about 6 kb, from about 6 kb to about 6.5 kb, from about 6.5 kb to about 7 kb, from about 7 kb to about 8 kb, from about 8 kb to about 9 kb, from about 9 kb to about 10 kb, from about 10 kb to about 11 kb, from about 11 kb to about 12 kb, from about 12 kb to about 13 kb, from about 13 kb to about 14 kb, or from about 14 kb to about 15 kb. In some cases, the heterologous nucleotide sequence has a length of from about 200 nt to about 1 kb. In some cases, the heterologous nucleotide sequence has a length of from about 1 kb to about 5 kb. In some cases, the heterologous nucleotide sequence has a length of from about 3.5 kb to about 6 kb. In some cases, the heterologous nucleotide sequence has a length of from about 6 kb to about 8 kb. In some cases, the heterologous nucleotide sequence has a length of from about 8 kb to about 15 kb. In some cases, the heterologous nucleotide sequence has a length of from about 9 kb to about 15 kb. In some cases, the heterologous nucleotide sequence has a length of from about 10 kb to about 15 kb.

### Methods of Delivering a Gene Product(s) to a Eukaryotic Host Cell

The present disclosure provides a method of delivering one or more gene products of interest to a eukaryotic cell, the method comprising contacting the cell with a gene delivery vehicle system of the present disclosure. The R2 polypeptide, the 5′ UTR, and the 3′ UTR provide for insertion of the heterologous nucleotide sequence into a 28S region of the genome of the eukaryotic cell.

In some cases, the eukaryotic cell is in vitro. In some cases, the eukaryotic cell is in vivo. In some cases, the eukaryotic cell is ex vivo.

Suitable eukaryotic cells include, e.g., a human cell, a non-human animal cell, a plant cell, a vertebrate cell, an invertebrate cell, a bird cell, an arthropod cell, an arachnid cell, an insect cell, a reptile cell, an amphibian cell, and the like. In some cases, the eukaryotic cell is a human cell. In some cases, the eukaryotic cell is a non-human animal cell. In some cases, the eukaryotic cell is a plant cell. In some cases, the eukaryotic cell is an invertebrate cell. In some cases, the cell is a diseased cell.

Suitable cells include a stem cell (e.g. an embryonic stem (ES) cell, an induced pluripotent stem (iPS) cell; a germ cell (e.g., an oocyte, a sperm, an oogonia, a spermatogonia, etc.); a somatic cell, e.g. a fibroblast, an oligodendrocyte, a glial cell, a hematopoietic cell, a neuron, a muscle cell, a bone cell, a hepatocyte, a pancreatic cell, etc.

Suitable cells include human embryonic stem cells, fetal cardiomyocytes, myofibroblasts, mesenchymal stem cells, cardiomyocytes, adipocytes, totipotent cells, pluripotent cells, blood stem cells, myoblasts, adult stem cells, bone marrow cells, mesenchymal cells, embryonic stem cells, parenchymal cells, epithelial cells, endothelial cells, mesothelial cells, fibroblasts, osteoblasts, chondrocytes, exogenous cells, endogenous cells, stem cells, hematopoietic stem cells, bone-marrow derived progenitor cells, myocardial cells, skeletal cells, fetal cells, undifferentiated cells, multi-potent progenitor cells, unipotent progenitor cells, monocytes, cardiac myoblasts, skeletal myoblasts, macrophages, capillary endothelial cells, xenogenic cells, allogenic cells, and post-natal stem cells. Suitable cells include peripheral blood mononuclear cells (PBMCs).

In some cases, the cell is an immune cell, a neuron, an epithelial cell, and endothelial cell, or a stem cell. In some cases, the immune cell is a T cell, a B cell, a monocyte, a natural killer cell, a dendritic cell, or a macrophage. In some cases, the immune cell is a cytotoxic T cell. In some cases, the immune cell is a helper T cell. In some cases, the immune cell is a regulatory T cell (Treg).

In some cases, the cell is a stem cell. Stem cells include adult stem cells. Adult stem cells are also referred to as somatic stem cells.

Adult stem cells are resident in differentiated tissue, but retain the properties of self-renewal and ability to give rise to multiple cell types, usually cell types typical of the tissue in which the stem cells are found. Numerous examples of somatic stem cells are known to those of skill in the art, including muscle stem cells; hematopoietic stem cells; epithelial stem cells; neural stem cells; mesenchymal stem cells; mammary stem cells; intestinal stem cells; mesodermal stem cells; endothelial stem cells; olfactory stem cells; neural crest stem cells; and the like.

Stem cells of interest include mammalian stem cells, where the term “mammalian” refers to any animal classified as a mammal, including humans; non-human primates; domestic and farm animals; and zoo, laboratory, sports, or pet animals, such as dogs, horses, cats, cows, mice, rats, rabbits, etc. In some cases, the stem cell is a human stem cell. In some cases, the stem cell is a rodent (e.g., a mouse; a rat) stem cell. In some cases, the stem cell is a non-human primate stem cell.

Stem cells can express one or more stem cell markers, e.g., SOX2, OCT4, NANOG, NESTIN, SOX1, PAX6, KLF4, SOX9, KRT19, KRT7, LGR5, CA9, FXYD2, CDH6, CLDN18, TSPAN8, BPIFB1, OLFM4, CDH17, and PPARGC1A.

In some embodiments, the stem cell is a hematopoietic stem cell (HSC). HSCs are mesoderm-derived cells that can be isolated from bone marrow, blood, cord blood, fetal liver and yolk sac. HSCs are characterized as CD34⁺ and CD3⁻. HSCs can repopulate the erythroid, neutrophil-macrophage, megakaryocyte and lymphoid hematopoietic cell lineages in vivo. In vitro, HSCs can be induced to undergo at least some self-renewing cell divisions and can be induced to differentiate to the same lineages as is seen in vivo. As such, HSCs can be induced to differentiate into one or more of erythroid cells, megakaryocytes, neutrophils, macrophages, and lymphoid cells.

In other embodiments, the stem cell is a neural stem cell (NSC). Neural stem cells (NSCs) are capable of differentiating into neurons, and glia (including oligodendrocytes, and astrocytes). A neural stem cell is a multipotent stem cell which is capable of multiple divisions, and under specific conditions can produce daughter cells which are neural stem cells, or neural progenitor cells that can be neuroblasts or glioblasts, e.g., cells committed to become one or more types of neurons and glial cells respectively. Methods of obtaining NSCs are known in the art.

In other embodiments, the stem cell is a mesenchymal stem cell (MSC). MSCs originally derived from the embryonal mesoderm and isolated from adult bone marrow, can differentiate to form muscle, bone, cartilage, fat, marrow stroma, and tendon. Methods of isolating MSC are known in the art; and any known method can be used to obtain MSC. See, e.g., U.S. Pat. No. 5,736,396, which describes isolation of human MSC.

A cell is in some cases a plant cell. A plant cell can be a cell of a monocotyledon. A cell can be a cell of a dicotyledon.

A cell is in some cases an arthropod cell. For example, the cell can be a cell of a sub-order, a family, a sub-family, a group, a sub-group, or a species of, e.g., Chelicerata, Myriapodia, Hexipodia, Arachnida, Insecta, Archaeognatha, Thysanura, Palaeoptera, Ephemeroptera, Odonata, Anisoptera, Zygoptera, Neoptera, Exopterygota, Plecoptera, Embioptera, Orthoptera, Zoraptera, Dermaptera, Dictyoptera, Notoptera, Grylloblattidae, Mantophasmatidae, Phasmatodea, Blattaria, Isoptera, Mantodea, Parapneuroptera, Psocoptera, Thysanoptera, Phthiraptera, Hemiptera, Endopterygota or Holometabola, Hymenoptera, Coleoptera, Strepsiptera, Raphidioptera, Megaloptera, Neuroptera, Mecoptera, Siphonaptera, Diptera, Trichoptera, or Lepidoptera.

A cell is in some cases an insect cell. For example, in some cases, the cell is a cell of a mosquito, a grasshopper, a true bug, a fly, a flea, a bee, a wasp, an ant, a louse, a moth, or a beetle.

A gene delivery system of the present disclosure can be introduced into a eukaryotic cell by any of a variety of methods, many of which are known in the art. Suitable methods include e.g., viral infection, transfection, lipofection, electroporation, calcium phosphate precipitation, polyethyleneimine (PEI)-mediated transfection, DEAE-dextran mediated transfection, liposome-mediated transfection, particle gun technology, calcium phosphate precipitation, direct microinjection, nanoparticle-mediated nucleic acid delivery, and the like.

Nucleic acids may be introduced into a eukaryotic cell using well-developed transfection techniques; see, e.g. Angel and Yanik (2010) PLoS ONE 5(7): e11756, and the commercially available TransMessenger® reagents from Qiagen, Stemfect™ RNA Transfection Kit from Stemgent, and TransIT®-mRNA Transfection Kit from Mirus Bio LLC. See also Beumer et al. (2008) PNAS 105(50):19821-19826.

In some cases, a gene delivery system of the present disclosure is administered to an individual in need thereof. A gene delivery system of the present disclosure can be administered to an individual by any of a variety of routes of administration. Conventional and pharmaceutically acceptable routes of administration include intratumoral, peritumoral, intramuscular, intratracheal, intracranial, subcutaneous, intradermal, topical application, intravenous, intraarterial, rectal, nasal, oral, and other enteral and parenteral routes of administration. Routes of administration may be combined, if desired, or adjusted depending upon the gene delivery system and/or the desired effect. A gene delivery system of the present disclosure can be administered in a single dose or in multiple doses.

In some cases, a gene delivery system of the present disclosure is administered intravenously. In some cases, a gene delivery system of the present disclosure is administered intramuscularly. In some cases, a gene delivery system of the present disclosure is administered locally. In some cases, a gene delivery system of the present disclosure is administered intratumorally. In some cases, a gene delivery system of the present disclosure is administered peritumorally. In some cases, a gene delivery system of the present disclosure is administered intracranially. In some cases, a gene delivery system of the present disclosure is administered subcutaneously.

In some cases, a target cell, or a population of target cells, is removed from (obtained from) an individual; the target cell, or population of target cells, is contacted ex vivo with a gene delivery system of the present disclosure, to generate a genetically modified target cell or genetically modified population of target cells; and the genetically modified target cell or genetically modified population of target cells is administered to the individual from whom the target cell or population of target cells was/were obtained. Thus, in some cases, a method of the present disclosure comprises: a) contacting a target cell or population of target cells ex vivo with a gene delivery system of the present disclosure, thereby generating a genetically modified target cell or genetically modified population of target cells, where the target cell or population of target cells were obtained from an individual in need of treatment; and b) administering the genetically modified target cell or genetically modified population of target cells to the individual, thereby treating the individual. In some cases, a method of the present disclosure comprises: a) obtaining a target cell or population of target cells from an individual; b) contacting the target cell or population of target cells ex vivo with a gene delivery system of the present disclosure, thereby generating a genetically modified target cell or genetically modified population of target cells; and c) administering the genetically modified target cell or genetically modified population of target cells to the individual. As one non-limiting example, the cells can be T cells; and the heterologous polypeptide(s) can be a CAR (e.g., a single polypeptide chain CAR; or a heterodimeric CAR). As another example, the cells can be diseased cells, and the heterologous gene products can be: i) an RNA-guided effector polypeptide such as a Cas9 polypeptide; ii) a guide RNA.

### Examples of Non-Limiting Aspects of the Disclosure

Aspects, including embodiments, of the present subject matter described above may be beneficial alone or in combination, with one or more other aspects or embodiments. Without limiting the foregoing description, certain non-limiting aspects of the disclosure numbered 1-33 are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:

Aspect 1. A gene delivery vehicle system comprising: a) a first nucleic acid and a second nucleic acid, wherein: i) the first nucleic acid comprises a nucleotide sequence encoding an R2 retrotransposon R2 polypeptide; and ii) the second nucleic acid comprises a heterologous nucleotide sequence encoding one or more heterologous gene products, wherein the heterologous nucleotide sequence is flanked by an R2 retrotransposon 3′ untranslated region (UTR) and an R2 retrotransposon 5′ UTR, and wherein the heterologous nucleotide sequence has a length of at least 200 nucleotides; or b) a polypeptide and a nucleic acid, wherein: i) the polypeptide is an R2 retrotransposon R2 polypeptide; and ii) the nucleic acid comprises a heterologous nucleotide sequence encoding one or more heterologous gene products, wherein the heterologous nucleotide sequence is flanked by an R2 retrotransposon 3′ UTR and an R2 retrotransposon 5′ UTR, and wherein the heterologous nucleotide sequence has a length of at least 200 nucleotides.

Aspect 2. The gene delivery vehicle system of aspect 1, wherein the R2 polypeptide comprises an amino acid sequence having at least 85% amino acid sequence identity to the amino acid sequence depicted in FIG. 7.

Aspect 3. The gene delivery vehicle system of aspect 1 or aspect 2, wherein the heterologous nucleotide sequence encodes a single heterologous gene product.

Aspect 4. The gene delivery vehicle system of aspect 3, wherein the single heterologous gene product is a polypeptide.

Aspect 5. The gene delivery vehicle system of aspect 3, wherein the single heterologous gene product is an RNA.

Aspect 6. The gene delivery vehicle system of aspect 1 or aspect 2, wherein the heterologous nucleotide sequence encodes at least a first heterologous gene product and a second heterologous gene product.

Aspect 7. The gene delivery vehicle system of aspect 6, wherein the first heterologous gene product is a polypeptide, and wherein the second heterologous gene product is an RNA.

Aspect 8. The gene delivery vehicle system of aspect 4, wherein the polypeptide is a chimeric antigen receptor.

Aspect 9. The gene delivery vehicle system of aspect 6, wherein the first heterologous gene product is a first heterologous polypeptide, and wherein the second heterologous gene product is a second heterologous polypeptide.

Aspect 10. The gene delivery vehicle system of aspect 9, wherein the heterologous nucleotide sequence comprises, in order from 5′ to 3′: i) a nucleotide sequence encoding the first heterologous polypeptide; ii) an internal ribosome entry site, or nucleotide sequence encoding a self-cleaving polypeptide; and iii) a nucleotide sequence encoding the second heterologous polypeptide.

Aspect 11. The gene delivery vehicle system of aspect 9, wherein the first polypeptide and the second polypeptide together form a heterodimeric chimeric antigen receptor.

Aspect 12. The gene delivery vehicle system of aspect 7, wherein the polypeptide is an RNA-guided effector polypeptide, and wherein the RNA is a guide RNA that binds to the RNA-guided effector polypeptide.

Aspect 13. The gene delivery vehicle system of any one of aspects 1-12, wherein the R2 polypeptide-encoding nucleotide sequence is codon optimized for expression in a eukaryotic cell.

Aspect 14. The gene delivery vehicle system of any one of aspects 1-13, wherein the heterologous nucleotide sequence encoding one or more heterologous gene products is operably linked to a transcriptional control element.

Aspect 15. The gene delivery vehicle system of aspect 14, wherein the transcriptional control element is a regulatable promoter.

Aspect 16. The gene delivery vehicle system of aspect 14, wherein the transcriptional control element is a constitutive promoter.

Aspect 17. The gene delivery vehicle system of any one of aspects 1-16, wherein the heterologous nucleotide sequence has a length of at least 3 kb.

Aspect 18. The gene delivery vehicle system of any one of aspects 1-16, wherein the heterologous nucleotide sequence has a length of from about 5 kb to about 10 kb.

Aspect 19. The gene delivery vehicle system of any one of aspects 1-16, wherein the heterologous nucleotide sequence has a length of from about 10 kb to about 15 kb.

Aspect 20. A kit comprising:

a1) a first nucleic acid comprising a nucleotide sequence encoding an R2 retrotransposon R2 polypeptide; and

b1) a second nucleic acid comprising an insertion site for inserting a heterologous nucleotide sequence encoding one or more heterologous gene products, wherein the insertion site is flanked by an R2 retrotransposon 3′ untranslated region (UTR) and an R2 retrotransposon 5′ UTR, and wherein the heterologous nucleotide sequence has a length at least 200 nucleotides; or

a2) an R2 retrotransposon R2 polypeptide; and

b2) a nucleic acid comprising an insertion site for inserting a heterologous nucleotide sequence encoding one or more heterologous gene products, wherein the insertion site is flanked by an R2 retrotransposon 3′ UTR and an R2 retrotransposon 5′ UTR, and wherein the heterologous nucleotide sequence has a length at least 200 nucleotides.

Aspect 21. The kit of aspect 20(a1 and b1), wherein the first nucleic acid and the second nucleic acid are in separate containers.

Aspect 22. The kit of aspect 20(a1), wherein the R2 polypeptide-encoding nucleotide sequence is codon optimized for expression in a eukaryotic cell.

Aspect 23. The kit of aspect 20(b1), wherein the second nucleic acid further comprises a transcriptional control element 5′ of the insertion site.

Aspect 24. The kit of aspect 20(a2 and b2), wherein the R2 polypeptide and the nucleic acid are in separate containers.

Aspect 25. The kit of aspect 20(b2), wherein the nucleic acid further comprises a transcriptional control element 5′ of the insertion site.

Aspect 26. A method of delivering one or more gene products of interest to a eukaryotic cell, the method comprising contacting the cell with the gene delivery vehicle system of any one of aspects 1-19, wherein the R2 polypeptide, the 5′ UTR, and the 3′ UTR provide for insertion of the heterologous nucleic acid into a 28S region of the genome of the eukaryotic cell.

Aspect 27. The method of aspect 26, wherein said contacting is in vitro.

Aspect 28. The method of aspect 26, wherein said contacting is in vivo.

Aspect 29. The method of aspect 26, wherein said contacting is ex vivo.

Aspect 30. The method of any one of aspects 26-29, wherein the eukaryotic cell is a non-human animal cell.

Aspect 31. The method of any one of aspects 26-29, wherein the eukaryotic cell is a human cell.

Aspect 32. The method of any one of aspects 26-29, wherein the eukaryotic cell is a plant cell.

Aspect 33. The method of any one of aspects 26-29, wherein the eukaryotic cell is an invertebrate cell.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

### Example 1

Two DNA plasmids, based off of the DNA from the silkworm Bombyx mori, were generated. One plasmid contains an optimized version of the coding sequence for the protein R2 (OR2Bm) while the second plasmid contains the flanking 5′ and 3′ untranslated regions (UTRs), a transgene oriented in the 3′ to 5′ direction, all under the control of the single rDNA promoter that controls transcription of the full rDNA transcript.

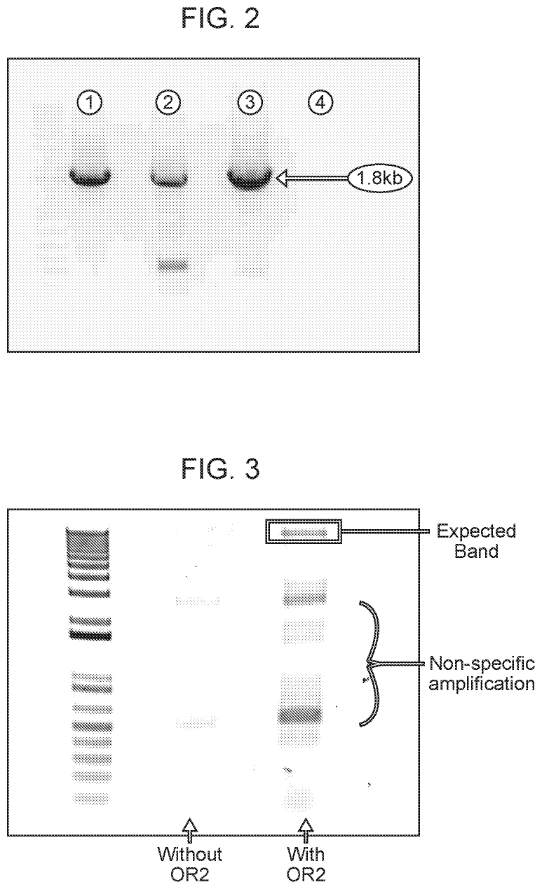

HEK 293 cells and HEK 293T cells were PEI transfected with both plasmid constructs and, after 48 hours, genomic DNA was extracted and sequenced through the 5′ junction of integration to ensure that both the transgene integrated at the correct site and that full-length integration occurred (FIG. 2). The transgene cassette was also expanded to include a hygromycin resistance gene and then used to quantify integration frequency and perform a titration to determine the most effective ratio. Transfection of the plasmid containing the UTRs and transgene yielded an integration efficiency of 1% through homologous-driven recombination (HDR). When co-transfected with the OR2 plasmid, the efficiency was increased to 4% of the total cell population.

FIG. 2: Genomic DNA was amplified across the integration junction in 28S rDNA and showed a specific band when the transgene flanked by 5′ and 3′ UTR under a CMV promoter was amplified with OR2Bm (lane 1) and without (lane 3). The transgene cassette under the control of an RNA pol I promoter (lane 2) showed a specific band of integration only in the presence of OR2Bm and none without (lane 4).

FIG. 3 shows that a full-length amplicon was amplified from genomic DNA without a band present when cells were only transfected with the transgene flanked by UTRs but without OR2.

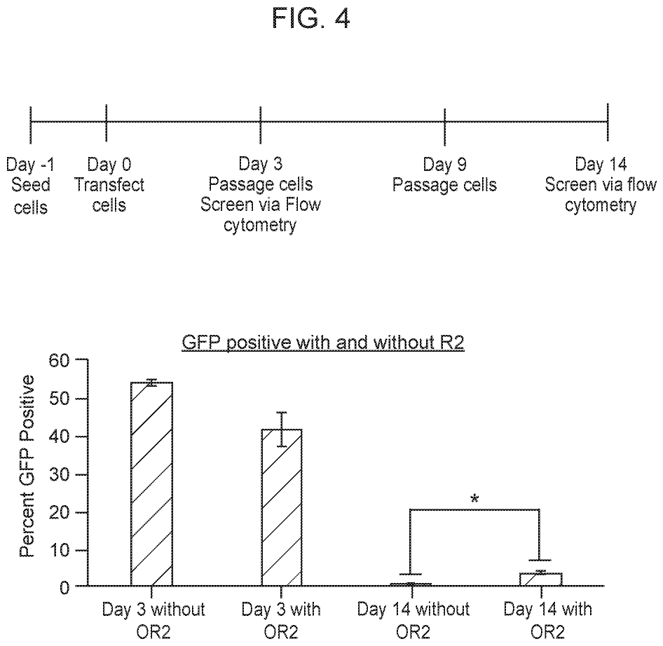

Cells were transfected with either the transgene flanked by UTRs or with the transgene flanked by UTRs and OR2. FIG. 4 schematically depicts the protocol. After three days, cells were passaged at a ratio of 1:10 and a portion was taken to be screened via flow cytometry. Cells were again passaged at a ratio of 1:10 six days later and at day 14, cells were screened via flow cytometry to analyze stable expression of transgene (GFP). The data are presented in FIG. 4. GFP expression was compared between the two samples and shows that OR2 helps mediate the creation of a stable population of 3.4% GFP positive cells. Cells transfected with OR2 had 4-fold more GFP expression. *indicates p<0.005

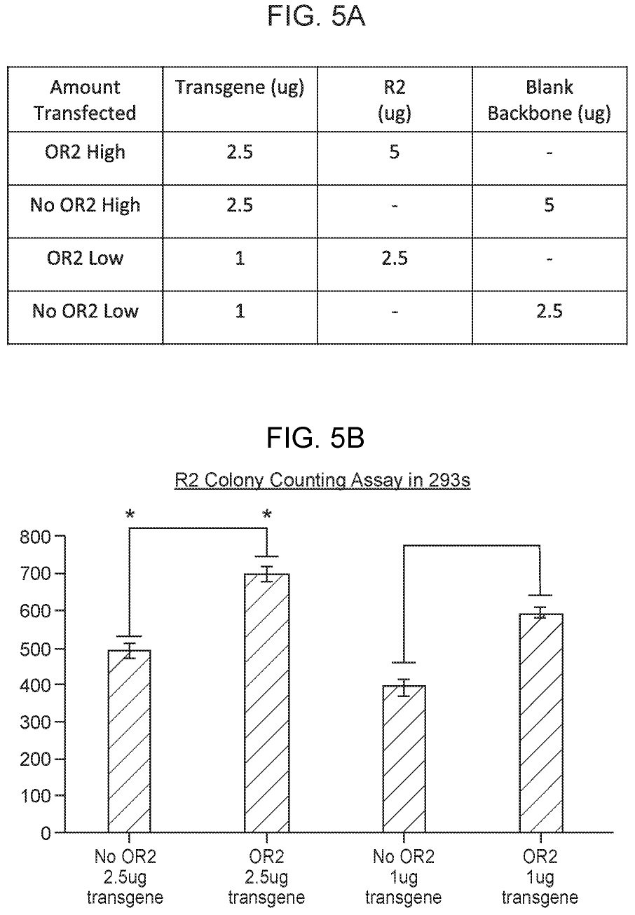

Cells were transfected at varying amounts of OR2 and transgene. The cells were then kept under antibiotic selection (200 μg/mL of Hygromycin B) for two weeks and the surviving colonies were counted after several wash. The data are presented in FIG. 5. As shown in FIG. 5, it was found that OR2 mediated a higher level of sustained expression. *indicates p<0.005

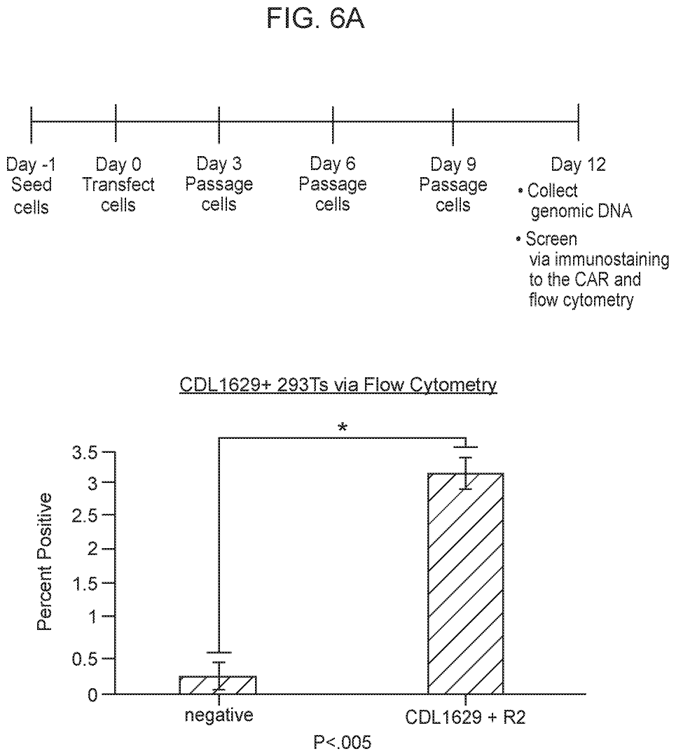

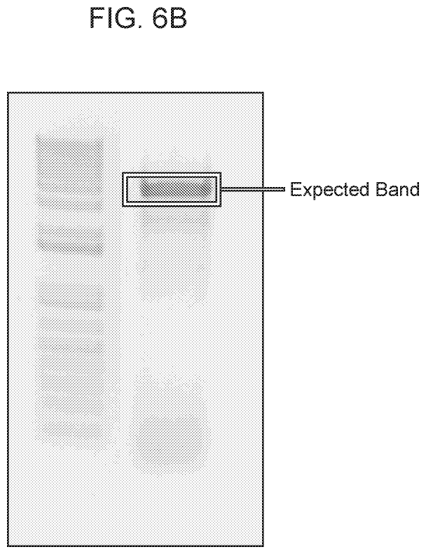

Cells were transfected with a CAR-T receptor and OR2 and passaged over two weeks. FIG. 6 schematically depicts the protocol. Afterwards, cells were labeled with an antibody against the receptor and screened via flow cytometry. Genomic DNA was then extracted and the full transcript was amplified to further confirm integration. The data are presented in FIG. 6. *indicates p<0.005.

### Example 2

Two gene delivery constructs are prepared. The first construct is a recombinant expression vector comprising a nucleotide sequence encoding an R2 retrotransposon R2 polypeptide. The second construct is a recombinant expression vector comprising a heterologous nucleotide sequence, oriented in the 3′ to 5′ direction, encoding 2 polypeptide chains of a conditionally active CAR. The heterologous nucleotide sequence is flanked by an R2 5′UTR and an R2 3′UTR. The R2 polypeptide is an optimized R2 (OR2) polypeptide. The first polypeptide chain of the conditionally active CAR comprises: i) an extracellular antigen binding domain that specifically binds to an antigen on a target cell (e.g., a cancer cell); ii) a transmembrane domain; and iii) a first member of a dimerization pair. The second polypeptide chain of the conditionally active CAR comprises: i) a transmembrane domain; ii) a second member of the dimerization pair; and iii) an intracellular signaling domain comprising an immunoreceptor tyrosine-based activation motif (ITAM), where the intracellular signaling domain provides signal transduction activity. The first polypeptide of the conditionally active CAR, the second polypeptide of the conditionally active CAR, or both the first and second polypeptides of the conditionally active CAR, comprises an intracellular costimulatory polypeptide.

The 2 gene delivery constructs are introduced into a T cell (e.g., a CD8⁺ T cell) in vitro, thereby genetically modifying the T cell. The genetically modified T cell is administered to an individual, e.g., an individual having a cancer, where the cancer comprises cells that express an antigen recognized by the conditionally active CAR.

### Example 3

Two gene delivery constructs are prepared. The first construct is a recombinant expression vector comprising a nucleotide sequence encoding an R2 retrotransposon R2 polypeptide. The second construct is a recombinant expression vector comprising a heterologous nucleotide sequence, oriented in the 3′ to 5′ direction, encoding a CAR and a synNotch polypeptide. The heterologous nucleotide sequence is flanked by an R2 5′UTR and an R2 3′UTR. The R2 polypeptide is an optimized R2 (OR2) polypeptide. The CAR is operably linked to a promoter that is activated by the intracellular signaling domain of the synNotch polypeptide.

### Example 4

Human embryonic kidney (HEK) 293 cells were transfected with: i) 2.5 μg of a plasmid containing a GFP expression construct (including a nucleotide sequence encoding GFP, where the nucleotide sequence is operably linked to a cytomegalovirus promoter) flanked by R2 5′UTR and R2 3′UTR, and oriented in the 3′-to-5′ direction relative to the UTRs; and ii) either 5 μg of a plasmid containing an expression cassette encoding OR2 or 5 μg of a plasmid containing stuffer DNA (control DNA that does not encode the OR2 polypeptide). The transfected HEK293 cells were then allowed to grow for 14 days to dilute and degrade unintegrated plasmid. After 14 days, the transfected HEK293 cells were quantified via flow cytometry to determine the stable population of cells that had integrated the GFP transgene.

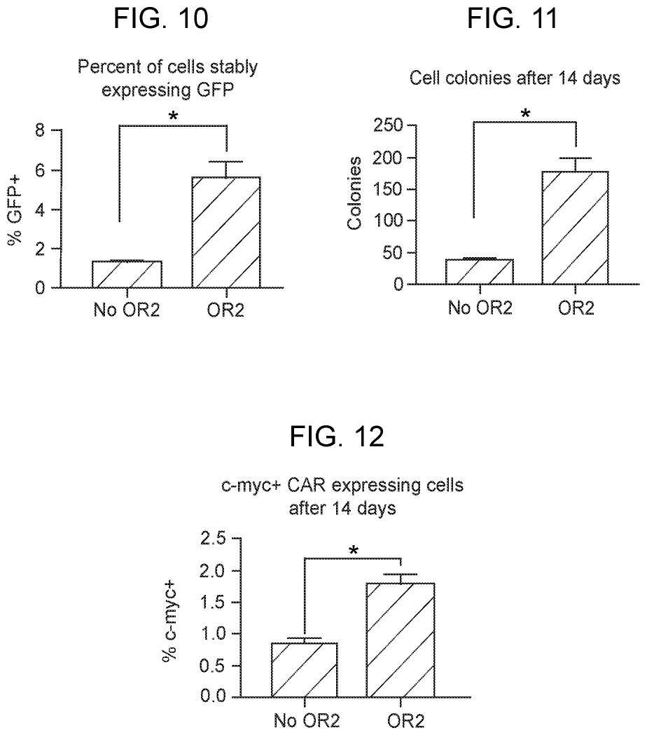

The results are shown in FIG. 10. On average, of the cells transfected with the construct containing OR2 and the GFP transgene, 5.6% stably expressed GFP. In contrast, on average, of the cells transfected with the stuffer and GFP transgene, 1.3% expressed GFP.

### Example 5

HEK293 cells were transfected with: i) 0.5 μg of a plasmid containing a HygromycinB (HygB) resistance cassette (including a nucleotide sequence encoding hygromycin B phosphotransferase, where the nucleotide sequence is operably linked to an SV40 promoter) flanked by R2 5′UTR and R2 3′UTR, and oriented in the 3′-to-5′ direction relative to the UTRs; and ii) either 0.5 μg of a plasmid containing an expression cassette encoding OR2 or 0.5 μg of a plasmid containing stuffer DNA (control DNA that does not encode the OR2 polypeptide). The transfected HEK293 cells were cultured in medium containing 200 μg/m hygromycin. The transfected HEK293 cells were allowed to grow for 14 days in the hygromycin-containing medium to dilute and degrade unintegrated plasmid. HEK293 cells were stained with methylene blue and then counted by hand to quantify the number of colonies, where each colony would represent one integration event.

The results are shown in FIG. 11. An average of 177 integration events occurred in cells transfected with OR2 and the HygB resistance cassette. An average of 39.33 integration events occurred in cells transfected with stuffer and the HygB resistance cassette.

### Example 6

HEK293 cells were transfected with: i) 2.5 μg of a plasmid containing a expression construct encoding a Chimeric Antigen Receptor (CAR) having a c-myc tag (CAR-c-myc) (where the expression construct comprises a nucleotide sequence encoding CAR-c-myc, where the nucleotide sequence is operably linked to an EF1-alpha promoter) flanked by the R2 untranslated regions (R2 5′UTR and R2 3′UTR), and oriented in the 3′-to-5′ direction relative to the UTRs; and ii) either 5 μg of a plasmid containing an expression cassette for OR2 or 5 μg of a plasmid containing stuffer DNA (control DNA that does not encode the OR2 polypeptide). The transfected HEK293 cells were then allowed to grow for 14 days to dilute and degrade unintegrated plasmid. After 14 days, the transfected HEK293 cells were quantified via flow cytometry by staining for cell surface expression of the c-myc tag to determine the stable population of cells that had integrated the transgene.

The data are shown in FIG. 12. On average, of the cells transfected with the construct containing OR2 and the CAR-c-myc transgene, 1.85% stably expressed the c-myc tag. In contrast, on average, of the cells transfected the stuffer and GFP transgene, 0.87% expressed the c-myc tag.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A gene delivery vehicle system comprising:
a) a first nucleic acid and a second nucleic acid, wherein:
i) the first nucleic acid comprises a nucleotide sequence encoding an R2 retrotransposon R2 polypeptide; and
ii) the second nucleic acid comprises a heterologous nucleotide sequence encoding one or more heterologous gene products, wherein the heterologous nucleotide sequence is flanked by an R2 retrotransposon 3′ untranslated region (UTR) and an R2 retrotransposon 5′ UTR, and wherein the heterologous nucleotide sequence has a length of at least 200 nucleotides; or
b) a polypeptide and a nucleic acid, wherein:
i) the polypeptide is an R2 retrotransposon R2 polypeptide; and
ii) the nucleic acid comprises a heterologous nucleotide sequence encoding one or more heterologous gene products, wherein the heterologous nucleotide sequence is flanked by an R2 retrotransposon 3′ UTR and an R2 retrotransposon 5′ UTR, and wherein the heterologous nucleotide sequence has a length of at least 200 nucleotides.

2. The gene delivery vehicle system of claim 1, wherein the R2 polypeptide comprises an amino acid sequence having at least 85% amino acid sequence identity to the amino acid sequence depicted in FIG. 7.

3. The gene delivery vehicle system of claim 1 or claim 2, wherein the heterologous nucleotide sequence encodes a single heterologous gene product.

4. The gene delivery vehicle system of claim 3, wherein the single heterologous gene product is a polypeptide.

5. The gene delivery vehicle system of claim 3, wherein the single heterologous gene product is an RNA.

6. The gene delivery vehicle system of claim 1 or claim 2, wherein the heterologous nucleotide sequence encodes at least a first heterologous gene product and a second heterologous gene product.

7. The gene delivery vehicle system of claim 6, wherein the first heterologous gene product is a polypeptide, and wherein the second heterologous gene product is an RNA.

8. The gene delivery vehicle system of claim 4, wherein the polypeptide is a chimeric antigen receptor.

9. The gene delivery vehicle system of claim 6, wherein the first heterologous gene product is a first heterologous polypeptide, and wherein the second heterologous gene product is a second heterologous polypeptide.

10. The gene delivery vehicle system of claim 9, wherein the heterologous nucleotide sequence comprises, in order from 5′ to 3′:
i) a nucleotide sequence encoding the first heterologous polypeptide;
ii) an internal ribosome entry site, or nucleotide sequence encoding a self-cleaving polypeptide; and
iii) a nucleotide sequence encoding the second heterologous polypeptide.

11. The gene delivery vehicle system of claim 9, wherein the first polypeptide and the second polypeptide together form a heterodimeric chimeric antigen receptor.

12. The gene delivery vehicle system of claim 7, wherein the polypeptide is an RNA-guided effector polypeptide, and wherein the RNA is a guide RNA that binds to the RNA-guided effector polypeptide.

13. The gene delivery vehicle system of any one of claims 1-12, wherein the R2 polypeptide-encoding nucleotide sequence is codon optimized for expression in a eukaryotic cell.

14. The gene delivery vehicle system of any one of claims 1-13, wherein the heterologous nucleotide sequence encoding one or more heterologous gene products is operably linked to a transcriptional control element.

15. The gene delivery vehicle system of claim 14, wherein the transcriptional control element is a regulatable promoter.

16. The gene delivery vehicle system of claim 14, wherein the transcriptional control element is a constitutive promoter.

17. The gene delivery vehicle system of any one of claims 1-16, wherein the heterologous nucleotide sequence has a length of at least 3 kb.

18. The gene delivery vehicle system of any one of claims 1-16, wherein the heterologous nucleotide sequence has a length of from about 5 kb to about 10 kb.

19. The gene delivery vehicle system of any one of claims 1-16, wherein the heterologous nucleotide sequence has a length of from about 10 kb to about 15 kb.

20. A kit comprising:
a1) a first nucleic acid comprising a nucleotide sequence encoding an R2 retrotransposon R2 polypeptide; and
b1) a second nucleic acid comprising an insertion site for inserting a heterologous nucleotide sequence encoding one or more heterologous gene products, wherein the insertion site is flanked by an R2 retrotransposon 3′ untranslated region (UTR) and an R2 retrotransposon 5′ UTR, and wherein the heterologous nucleotide sequence has a length at least 200 nucleotides; or
a2) an R2 retrotransposon R2 polypeptide; and
b2) a nucleic acid comprising an insertion site for inserting a heterologous nucleotide sequence encoding one or more heterologous gene products, wherein the insertion site is flanked by an R2 retrotransposon 3′ UTR and an R2 retrotransposon 5′ UTR, and wherein the heterologous nucleotide sequence has a length at least 200 nucleotides.

21. The kit of claim 20(a1 and b1), wherein the first nucleic acid and the second nucleic acid are in separate containers.

22. The kit of claim 20(a1), wherein the R2 polypeptide-encoding nucleotide sequence is codon optimized for expression in a eukaryotic cell.

23. The kit of claim 20(b1), wherein the second nucleic acid further comprises a transcriptional control element 5′ of the insertion site.

24. The kit of claim 20(a2 and b2), wherein the R2 polypeptide and the nucleic acid are in separate containers.

25. The kit of claim 20(b2), wherein the nucleic acid further comprises a transcriptional control element 5′ of the insertion site.

26. A method of delivering one or more gene products of interest to a eukaryotic cell, the method comprising contacting the cell with the gene delivery vehicle system of any one of claims 1-19, wherein the R2 polypeptide, the 5′ UTR, and the 3′ UTR provide for insertion of the heterologous nucleic acid into a 28S region of the genome of the eukaryotic cell.

27. The method of claim 26, wherein said contacting is in vitro.

28. The method of claim 26, wherein said contacting is in vivo.

29. The method of claim 26, wherein said contacting is ex vivo.

30. The method of any one of claims 26-29, wherein the eukaryotic cell is a non-human animal cell.

31. The method of any one of claims 26-29, wherein the eukaryotic cell is a human cell.

32. The method of any one of claims 26-29, wherein the eukaryotic cell is a plant cell.

33. The method of any one of claims 26-29, wherein the eukaryotic cell is an invertebrate cell.
